(19) United States
(12) Patent Application Publication
(10) Pub. No.: US 2021/0340621 A1
(43) Pub. Date: Nov. 4, 2021

(54) METHODS AND SYSTEMS FOR SEQUENCING

(71) Applicant: ReadCoor, LLC, Pleasanton, CA (US)

(72) Inventors: Evan Daugharthy, Cambridge, MA (US); Richard Terry, Carlisle, MA (US)

(21) Appl. No.: 17/235,625

(22) Filed: Apr. 20, 2021

Related U.S. Application Data
(63) Continuation of application No. 63/013,913, filed on Apr. 22, 2020.

(51) Int. Cl. C12Q 1/6876; C12Q 1/682; C12Q 1/6844; C12Q 1/6809

(52) U.S. Cl. CPC C12Q 1/6876; C12Q 1/6809; C12Q 1/6844; C12Q 1/682

(57) ABSTRACT

Provided herein are compositions, methods, and systems for amplifying and identifying nucleic acids within a biological sample. The compositions, methods, and systems are generally compatible with volumetric imaging techniques and samples comprising nucleic acids contained within a three-dimensional matrix.

### CROSS-REFERENCE

This application claims the benefit of U.S. Provisional Patent Application Ser. No. 63/013,913, filed Apr. 22, 2020, which is entirely incorporated herein by reference.

### SEQUENCE LISTING

The instant application contains a Sequence Listing which has been submitted electronically in ASCII format and is hereby incorporated by reference in its entirety. Said ASCII copy, created on Jun. 23, 2021, is named 52160-732_201_SL.txt and is 3,899 bytes in size.

### BACKGROUND

Determining the order of nucleic acid residues in biological samples is an integral component of a wide variety of research applications. A variety of techniques and technologies have been developed to facilitate this feat, sequencing deoxyribonucleic acid (DNA) and/or ribonucleic acid (RNA) molecules. Tremendous changes have occurred, moving from sequencing short oligonucleotides to millions of bases, from struggling towards the deduction of the coding sequence of a single gene to rapid and widely available whole genome sequencing.

In situ sequencing, e.g., fluorescent in situ sequencing (FISSEQ), can be used to detect target molecules while they remain in a sample (e.g., a cell or a tissue). During FISSEQ, a three-dimensional (3D) matrix can be generated within the sample to immobilize the target molecules or derivatives thereof. Nucleic acid target molecules may be subsequently amplified and sequenced within the 3D matrix. The 3D matrix with the attached nucleic acid molecules can provide an information storage medium where the nucleic acid molecules represent stored information which can be read within the 3D matrix.

### SUMMARY

The present disclosure provides compositions, methods and systems for determining the sequence of nucleotides in a target nucleic acid molecule using sequencing by ligation and/or sequencing by hybridization. The present disclosure also provides compositions, methods and systems for amplifying signals for detection during sequencing. A primary probe may be used to bind to the target nucleic acid molecule, and signal amplification may be achieved through secondary amplification upon binding of a secondary probe to the primary probe. Certain aspects include repeated cycles of duplex extension along a nucleic acid template, such as a single stranded nucleic acid template, using probes that facilitate detection of one or more or all of the nucleotides in an oligonucleotide probe that is hybridized and/or ligated in duplex extension to the nucleic acid template. Compositions and methods provided herein can be used in in situ sequencing. For example, the compositions and methods provided herein can be used to sequence a target nucleic acid molecule immobilized within a three-dimensional (3D) matrix.

In an aspect, the present disclosure provides a method for identifying one or more nucleotides of a nucleic acid molecule, comprising: (a) providing the nucleic acid molecule having hybridized thereto a sequencing primer and at least one nucleic acid probe, wherein the at least one nucleic acid probe comprises (i) a template hybridizing sequence that is complementary to a sequence of the nucleic acid molecule and (ii) a template nonhybridizing sequence, which template nonhybridizing sequence corresponds to the one or more nucleotides of the nucleic acid molecule, and wherein the template nonhybridizing sequence comprises a nucleic acid initiator; (b) ligating said sequencing primer to said at least one nucleic acid probe; (c) contacting the nucleic acid molecule with a plurality of nucleic acid amplifiers such that the nucleic acid initiator of the template nonhybridizing sequence initiates an amplification reaction of at least a subset of the plurality of nucleic acid amplifiers to form an amplification product attached to the nucleic acid initiator; (d) detecting a signal from the amplification product to identify one or more other nucleotides of the template nonhybridizing sequence, which one or more other nucleotides corresponds to the one or more nucleotides of the nucleic acid molecule; and (e) using at least the one or more other nucleotides identified in (d) to identify the one or more nucleotides of the nucleic acid molecule.

In some embodiments, the amplification reaction is a hybridization chain reaction (HCR), and wherein a nucleic acid amplifier of the plurality of nucleic acid amplifiers is an HCR monomer. In some embodiments, the HCR monomer is a metastable nucleic acid hairpin. In some embodiments, the HCR monomer comprises a detectable label. In some embodiments, the detectable label is attached to the HCR monomer through a linker. In some embodiments, the linker is a cleavable linker. In some embodiments, the cleavable linker is a disulfide bond. In some embodiments, the method further comprises, subsequent to (e), cleaving the cleavable linker, thereby cleaving the detectable label from the HCR monomer. In some embodiments, the amplification reaction is a branched nucleic acid amplification, and wherein the nucleic acid initiator is attached to the amplification product through a preamplifier sequence. In some embodiments, a nucleic acid amplifier of the plurality of nucleic acid amplifiers comprises a first portion that is complementary to the preamplifier sequence and a second portion that is not hybridizable to the preamplifier sequence. In some embodiments, the second portion of the nucleic acid amplifier further comprises a detectable label. In some embodiments, the detectable label is attached to the second portion of the nucleic acid amplifier. In some embodiments, the method further comprises contacting the second portion of the nucleic acid amplifier with a probe comprising the detectable label. In some embodiments, the second portion of the nucleic acid hybridizes to the probe comprising the detectable label. In some embodiments, the nucleic acid molecule is in a sample, and wherein (c) or (d) is performed while the nucleic acid molecule is in the sample. In some embodiments, the sample is a cell or a tissue. In some embodiments, the sample is fixed. In some embodiments, the sample is permeabilized. In some embodiments, the sample comprises a three-dimensional (3D) matrix. In some embodiments, the 3D matrix is a synthetic 3D matrix. In some embodiments, the sample is immobilized on a surface. In some embodiments, the nucleic acid molecule is immobilized on a surface. In some embodiments, the signal is a fluorescent signal. In some embodiments, the signal is generated by a plurality of fluorophores. In some embodiments, the signal is an optical signal. In some embodiments, the signal is an electrical signal or an electrochemical signal. In some embodiments, the electrical signal is a conductivity signal, impedance signal, or a charge signal. In some embodiments, the method further comprises removing the signal from the amplification product or from the nucleic acid molecule. In some embodiments, the removing is performed with aid of a reducing agent. In some embodiments, the template hybridizing sequence is cleavably attached to the template nonhybridizing sequence. In some embodiments, the template hybridizing sequence is cleavably attached to the template nonhybridizing sequence via a cleavable linker. In some embodiments, the cleavable linker is a photocleavable linker or a chemically cleavable linker. In some embodiments, the method further comprises, subsequent to (e), removing the template nonhybridizing sequence from the template hybridizing sequence to generate an extendable terminus on the template hybridizing sequence. In some embodiments, the method further comprises, after the removing the template nonhybridizing sequence, repeating (c) to (e) with an additional nucleic acid probe having a template hybridizing sequence having a sequence that is complementary with the nucleic acid sequence and a template nonhybridizing sequence. In some embodiments, (b) to (e) are repeated with an additional nucleic acid probe having a template hybridizing sequence having a sequence that is complementary with said nucleic acid sequence and a template nonhybridizing sequence.

In another aspect, the present disclosure provides a composition comprising: a nucleic acid molecule having hybridized thereto a sequencing primer and at least one nucleic acid probe hybridized thereto, wherein the at least one nucleic acid probe comprises (i) a template hybridizing sequence that is complementary to a sequence of the nucleic acid molecule and (ii) a template nonhybridizing sequence, which template nonhybridizing sequence corresponds to one or more nucleotides of the nucleic acid molecule, wherein the template nonhybridizing sequence comprises a nucleic acid initiator, wherein said sequencing primer is ligated to said at least one nucleic acid probe; and a plurality of nucleic acid amplifiers, and wherein at least a subset of the plurality of nucleic acid amplifiers is configured to form an amplification product attached to the nucleic acid initiator.

In another aspect, the present disclosure provides a kit for identifying one or more nucleotides of a nucleic acid molecule, comprising: a sequencing primer; at least one nucleic acid probe, wherein the at least one nucleic acid probe comprises (i) a template hybridizing sequence that is complementary to a sequence of the nucleic acid molecule and (ii) a template nonhybridizing sequence, which template nonhybridizing sequence corresponds to the one or more nucleotides of the nucleic acid molecule, and wherein the template nonhybridizing sequence comprises a nucleic acid initiator; a plurality of nucleic acid amplifiers; and instructions that direct a user to: (a) provide the nucleic acid molecule having hybridized thereto the sequencing primer and the at least one nucleic acid probe; (b) ligate said sequencing primer to said at least one nucleic acid probe; (c) contact the nucleic acid molecule with the plurality of nucleic acid amplifiers such that the nucleic acid initiator of the template nonhybridizing sequence initiates an amplification reaction of at least a subset of the plurality of nucleic acid amplifiers to form an amplification product attached to the nucleic acid initiator; (d) detect a signal from the amplification product to identify one or more other nucleotides of the template nonhybridizing sequence, which one or more other nucleotides corresponds to the one or more nucleotides of the nucleic acid molecule; and (e) use at least the one or more other nucleotides identified in (d) to identify the one or more nucleotides of the nucleic acid molecule.

In another aspect, the present disclosure provides a non-transitory computer-readable medium comprising machine-executable code that, upon execution by one or more computer processors, implements a method for identifying one or more nucleotides of a nucleic acid molecule, the method comprising: (a) providing the nucleic acid molecule having hybridized thereto a sequencing primer and at least one nucleic acid probe, wherein the at least one nucleic acid probe comprises (i) a template hybridizing sequence that is complementary to a sequence of the nucleic acid molecule and (ii) a template nonhybridizing sequence, which template nonhybridizing sequence corresponds to the one or more nucleotides of the nucleic acid molecule, and wherein the template nonhybridizing sequence comprises a nucleic acid initiator; (b) contacting the nucleic acid molecule with a plurality of nucleic acid amplifiers such that the nucleic acid initiator of the template nonhybridizing sequence initiates an amplification reaction of at least a subset of the plurality of nucleic acid amplifiers to form an amplification product attached to the nucleic acid initiator; (c) detecting a signal from the amplification product to identify one or more other nucleotides of the template nonhybridizing sequence, which one or more other nucleotides corresponds to the one or more nucleotides of the nucleic acid molecule; and (d) using at least the one or more other nucleotides identified in (c) to identify the one or more nucleotides of the nucleic acid molecule.

In another aspect, the present disclosure provides a system for identifying one or more nucleotides of a nucleic acid molecule, comprising: a support configured to hold the nucleic acid molecule having hybridized thereto a sequencing primer and at least one nucleic acid probe, wherein the at least one nucleic acid probe comprises (i) a template hybridizing sequence that is complementary to a sequence of the nucleic acid molecule and (ii) a template nonhybridizing sequence, which template nonhybridizing sequence corresponds to the one or more nucleotides of the nucleic acid molecule, and wherein the template nonhybridizing sequence comprises a nucleic acid initiator; a detector in sensing communication with the substrate; and one or more computer processors operatively coupled to the detector, wherein the one or more computer processors are individually or collectively programmed to direct: (a) contacting the nucleic acid molecule with a plurality of nucleic acid amplifiers such that the nucleic acid initiator of the template nonhybridizing sequence initiates an amplification reaction of at least a subset of the plurality of nucleic acid amplifiers to form an amplification product attached to the nucleic acid initiator; (b) using the detector to detect a signal from the amplification product to identify one or more other nucleotides of the template nonhybridizing sequence, which one or more other nucleotides corresponds to the one or more nucleotides of the nucleic acid molecule; and (c) using at least the one or more other nucleotides identified in (b) to identify the one or more nucleotides of the nucleic acid molecule.

Another aspect of the present disclosure provides a non-transitory computer-readable medium comprising machine-executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example sequencing scheme of using a primary probe with a template nonhybridizing sequence and a secondary probe attached to a detectable label for detection.

FIG. 2 shows an example sequencing scheme of using a primary probe with a template nonhybridizing sequence. The template nonhybridizing sequence can initiate an amplification reaction to amplify a signal upon binding of one or more secondary probes. In this example, a hybridization chain reaction (HCR) is initiated via the template nonhybridizing sequence of the primary probe to amplify a detection signal.

FIG. 3 shows an example sequencing scheme of using a primary probe with a template nonhybridizing sequence. The template nonhybridizing sequence can initiate an amplification reaction to amplify a signal upon binding of one or more secondary probes. In this example, a branched DNA (bDNA) reaction is initiated via the template nonhybridizing sequence of the primary probe to amplify a detection signal.

FIG. 4 shows an example sequencing scheme of using a primary probe with a template nonhybridizing sequence and reversable hybridization chain reaction (HCR) for detection. In this example, the detectable label is linked to the HCR monomer through a reversable (e.g., cleavable) linker, a disulfide bond. The disulfide bond can be cleaved by using a reducing buffer (e.g., tris(2-carboxyethyl)phosphine, TCEP buffer) to remove the signal.

FIG. 5 shows an example method of sequencing provided in the present disclosure.

FIG. 6 shows an example hybridization chain reaction (HCR) scheme.

FIG. 7 shows a computer system that is programmed or otherwise configured to implement methods provided herein.

FIG. 8A shows unamplified fluorescent signals detected via scanning confocal microscopy (AUTOLEVELED Image Adjustment; Rolony Signal ˜4,000 counts). The image depicts fluorescent secondary hybridization to non-template hybridizing region. FIG. 8B shows fluorescent signals amplified with 2×5 brDNA detected via scanning confocal microscopy (AUTOLEVELED Image Adjustment; Rolony Signal ˜40,000 counts).

FIG. 9A shows unamplified fluorescent signals detected via scanning confocal microscopy (Scaled Image Adjustment/100 min (Black value)/30,000 max (white value) in counts; Rolony Signal ˜4,000 counts). The image depicts fluorescent secondary hybridization to non-template hybridizing region. FIG. 9B shows fluorescent signals amplified with 2×5 brDNA detected via scanning confocal microscopy (Scaled Image Adjustment/100 min (Black value)/30,000 max (white value) in counts; Rolony Signal ˜40,000 counts).

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Alternatively, particularly with respect to biological systems or processes, the term may mean within an order of magnitude, within 5-fold, or within 2-fold, of a value. Where particular values are described in the application and claims, unless otherwise stated the term “about” meaning within an acceptable error range for the particular value may be assumed.

The term “nucleic acid,” as used herein, generally refers to a nucleic acid molecule comprising a plurality of nucleotides or nucleotide analogs. A nucleic acid may be a polymeric form of nucleotides. A nucleic acid may comprise deoxyribonucleotides and/or ribonucleotides, or analogs thereof. A nucleic acid may be an oligonucleotide or a polynucleotide. Nucleic acids may have various three-dimensional structures and may perform various functions. Non-limiting examples of nucleic acids include DNA, RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs, such as LNA or PNA. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation, with a functional moiety for immobilization.

The term “sticky end,” as used herein, refers to a nucleic acid sequence that is available to hybridize with a complementary nucleic acid sequence. The secondary structure of the “sticky end” is such that the sticky end is available to hybridize with a complementary nucleic acid under the appropriate reaction conditions without undergoing a conformational change. The sticky end can be a single stranded nucleic acid.

The term “monomer,” as used herein, refers to a nucleic acid oligomer. In some cases, at least two monomers are used in hybridization chain reactions (HCRs), although three, four, five, six or more monomers may be used. In some cases, more than two monomers are utilized, such as in the HCR systems displaying quadratic and exponential growth.

The term “metastable,” as used herein, means that in the absence of an initiator the monomers are kinetically disfavored from associating with other monomers comprising complementary regions. HCR monomers can be metastable monomers, which are able to assemble upon exposure to an initiator nucleic acid to form a HCR polymer.

The term “polymerization,” as used herein, refers to the association of two or more monomers to form a polymer. The “polymer” may comprise covalent bonds, non-covalent bonds or both. For example, in some cases, two species of monomers are able to hybridize in an alternating pattern to form a polymer comprising a nicked double helix. The polymer can also be referred to herein as “HCR polymer.”

The term “initiator,” as used herein, refers to a molecule or sequence of the molecule that is able to initiate the polymerization of monomers in HCRs or initiate the branched nucleic acid amplification. The initiator can comprise a nucleic acid region that is complementary to the initiator complement region of an HCR monomer. The initiator can comprise a nucleic acid region that is complementary to a preamplifier sequence or molecule.

The term “hybridization,” as used herein, refers to the process in which two single-stranded polynucleotides bind noncovalently to form a stable double-stranded polynucleotide. The term “hybridization” may also refer to triple-stranded hybridization. The resulting double-stranded polynucleotide is a “hybrid” or “duplex.”

### Methods of Detection

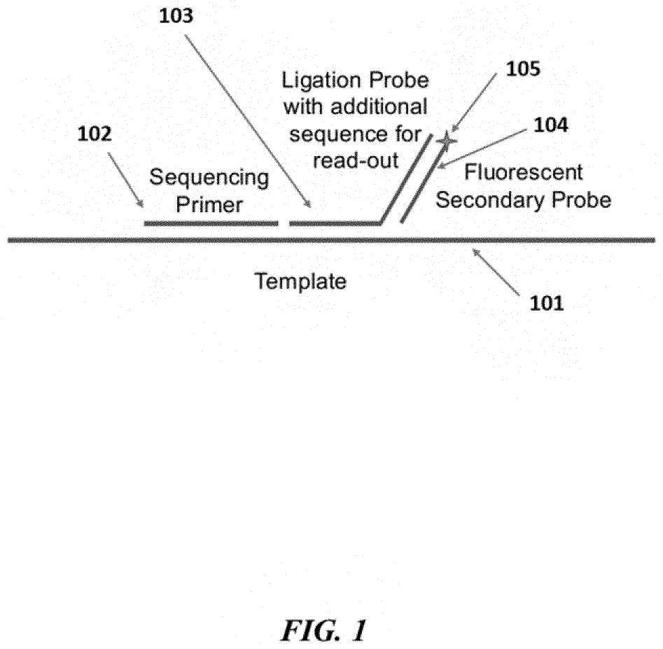

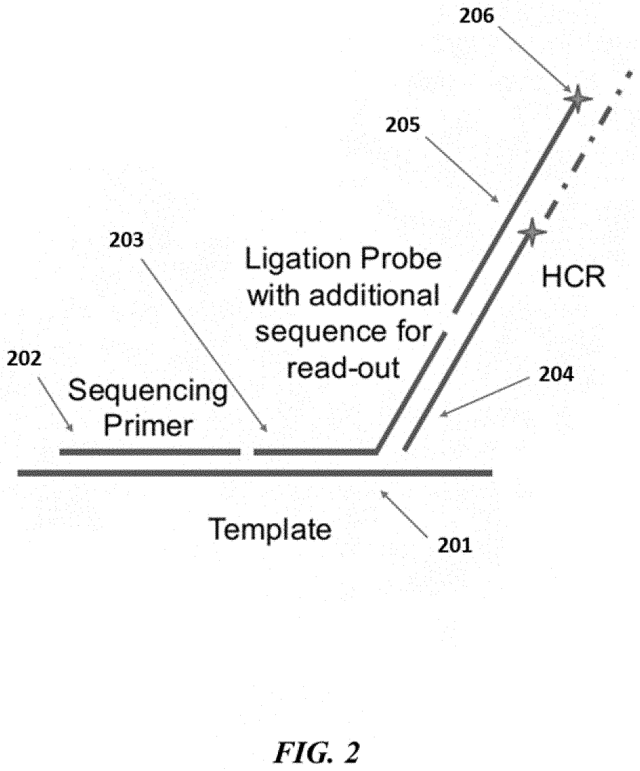

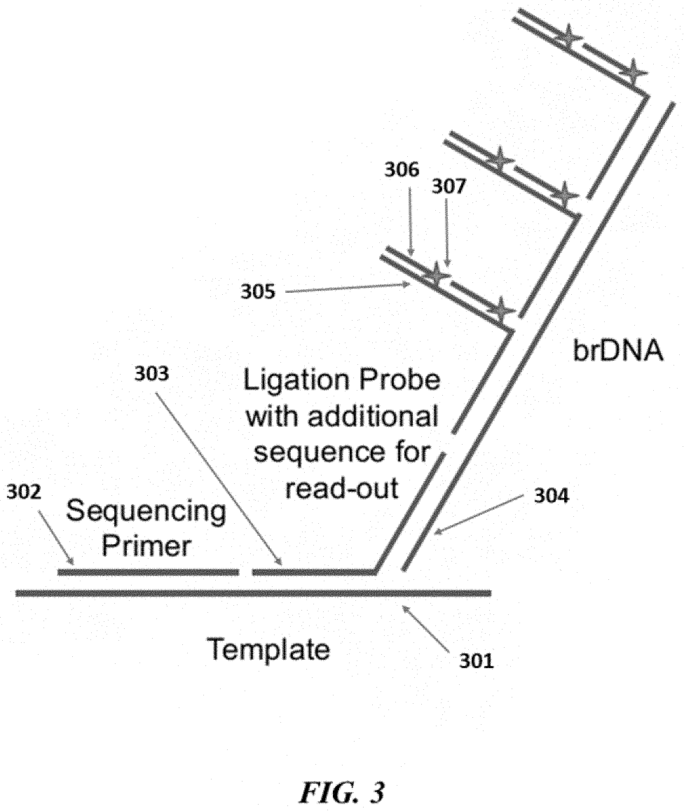

The present disclosure provides a method for identifying one or more nucleotides of a nucleic acid molecule (e.g., a target or template nucleic acid molecule) in a sample. The methods provided herein use a primary probe to bind to the target nucleic acid molecule and a secondary probe or plurality of secondary probes for amplifying a signal for detection. The amplification through the secondary probes can be referred to as secondary amplification. FIG. 1 provides an example scheme of using a secondary probe attached to a detectable label for detection of the primary probe. In this example, a template 101 is bound to a sequencing primer 102 and a primary probe 103 (e.g., a nucleic acid probe) having a template hybridizing sequence and a template nonhybridizing sequence (e.g., the additional sequence for read-out). The template nonhybridizing sequence can bind to a secondary probe 104 which can be further linked to a detectable label 105 for detection. The number of detectable units can be increased by secondary amplification to augment the signal for detection. FIG. 2 and FIG. 3 provide example schemes of the secondary amplification.

The method can comprise providing a sample comprising a nucleic acid molecule (e.g., a template or target nucleic acid molecule) comprising a sequencing primer and at least one nucleic acid probe hybridized to the nucleic acid molecule. The sequencing primer can hybridize to the nucleic acid molecule before, after, or simultaneously with the hybridization of the nucleic acid probe to the nucleic acid molecule. The at least one nucleic acid probe can comprise a template hybridizing sequence that is complementary to a sequence of the nucleic acid molecule. The at least one nucleic acid probe can further comprise a template nonhybridizing sequence. The template nonhybridizing sequence can correspond to one or more nucleotides of the nucleic acid molecule to be identified. For example, the template nonhybridizing sequence can correspond to one or more nucleotides of the template hybridizing sequence of the at least one nucleic acid probe, and the one or more nucleotides of the template hybridizing sequence in turn corresponds to one or more nucleotides of the nucleic acid molecule to be identified through sequence complementarity. In this way, detection of the template nonhybridizing sequence can identify the one or more nucleotides of the nucleic acid molecule (e.g., a target or template nucleic acid molecule).

The template nonhybridizing sequence can comprise a nucleic acid initiator. The nucleic acid initiator can be a sequence of the template nonhybridizing sequence, which may be used initiate a reaction to amplify a signal through nucleic acid hybridization or self-assembly. The reaction can be a non-enzymatic reaction or an enzymatic reaction. Next, the sample can be contacted with a plurality of nucleic acid amplifiers. The nucleic acid initiator of the template nonhybridizing sequence can initiate an amplification reaction of the plurality of nucleic acid amplifiers. In some cases, at least a subset of the plurality of nucleic acid amplifiers forms an amplification product attached to the nucleic acid initiator. Next, a signal from the amplification product can be detected to identify the template nonhybridizing sequence corresponding to the one or more nucleosides of the nucleic acid molecule, thereby identifying the one or more nucleotides of the nucleic acid molecule.

The amplification reaction can be a signal amplification by exchange reaction (SABER). A single stranded DNA primer can be extended by catalytic hairpins. A primer with a domain A on the 3′ end of the primer can bind to a catalytic hairpin and be extended with a new A domain by a strand displacing polymerase. Competitive branch migration can displace the newly extended primer which can then dissociate. The cycle can repeat and result in the generation of long concatemeric sequences. The length of concatemeric sequences can be controlled by, for example, hairpin concentration, polymerase concentration, and incubation time. When concatemers are bound to a target nucleic acid, concatemers can act as scaffolds to which multiple fluorescent strands can bind, thus serving as a platform to amplify signal of a nucleic acid probe.

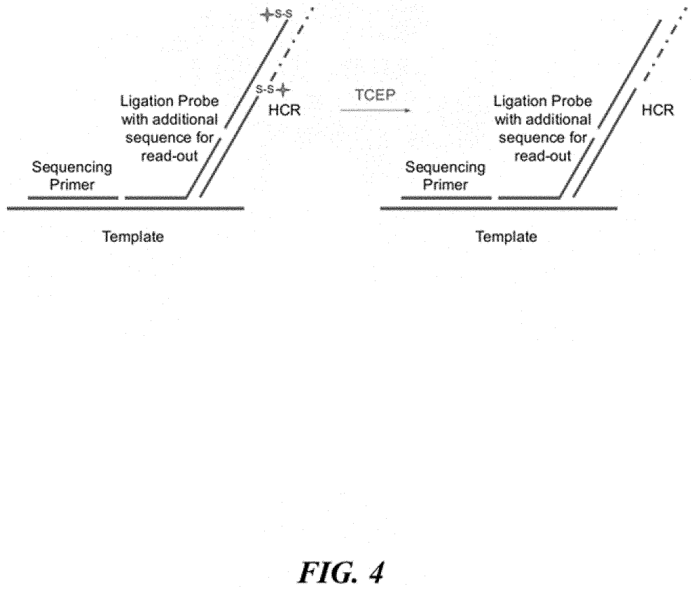

The amplification reaction may be a hybridization chain reaction (HCR) (see FIG. 2). Each nucleic acid amplifier of the plurality of nucleic acid amplifiers used during HCR can be an HCR monomer. The HCR monomer can be a metastable nucleic acid hairpin. The HCR monomer can comprise a detectable label. The detectable label can be attached to the HCR monomer. The detectable label can be attached to the HCR monomer covalently or non-covalently. The detectable label can be removably attached to the HCR monomer. In some cases, the detectable label can be attached to the HCR monomer through a linker (e.g., one or more chemical bonds). The linker can be a cleavable linker. The linker can be a chemically labile, enzymatically labile, or photolabile linker. In some embodiments, the detectable label can be attached to the HCR monomer via a bond that may be cleaved by exposure to reducing agents (e.g., a reducing buffer). For example, the cleavable linker can be a disulfide bond, and reduction of the disulfide bond can be used to remove the detectable label (see FIG. 4). The detectable label can be removed, released, or inactivated after detection. For example, the detectable label can be linked to the HCR monomer through a cleavable linker, and subsequent to detection of the signal, the cleavable linker can be cleaved, thereby cleaving the detectable label from the HCR monomer. For another example, in the cases where the detectable label is a fluorescent label, the fluorescent label can be quenched.

FIG. 2 shows an example sequencing scheme of using a primary probe with a template nonhybridizing sequence. In this example, the template 201 is bound to a sequencing primer 202 and a primary probe 203 (e.g., a nucleic acid probe) having a template hybridizing sequence and a template nonhybridizing sequence. The template nonhybridizing sequence can initiate an amplification reaction to amplify a signal upon binding of one or more secondary probes 204 and 205. In this example, a hybridization chain reaction (HCR) is initiated via the template nonhybridizing sequence of the primary probe 203 to amplify a detection signal. Two different HCR monomers 204 and 205, which have different sequences, can be used to generate a HCR polymer for signal amplification. One or more copies of each HCR monomer can be used to continue the HCR (as shown in dotted line). Each of the HCR monomers can be linked to a detectable label 206.

The amplification reaction may be a branched nucleic acid amplification (see FIG. 3). In these cases, the nucleic acid initiator can be attached to the amplification product through a preamplifier sequence. In the branched nucleic acid amplification, each nucleic acid amplifier of the plurality of nucleic acid amplifiers can comprise a first portion that is complementary to the preamplifier sequence and a second portion that is not hybridizable to the preamplifier sequence. The second portion of the nucleic acid amplifier may further comprise a detectable label. The detectable label can be attached to the second portion of the nucleic acid amplifier. Next, the second portion of the nucleic acid amplifier can be contacted with a probe comprising the detectable label. The second portion of the nucleic acid can hybridize to the probe comprising the detectable label. The detectable label can be removably attached to the probe. In some cases, the detectable label can be attached to the probe through a linker (e.g., one or more chemical bonds). The linker can be a cleavable linker. The linker can be a chemically labile, enzymatically labile, or photolabile linker. For example, the cleavable linker can be a disulfide bond, and reduction of the disulfide bond can be used to remove the detectable label from the probe. The detectable label can be removed after detection. For example, the detectable label can be removed by reversing the hybridization between the probe and the second portion of the nucleic acid amplifier or by cleaving a cleavable linker through which the detectable label is attached to the probe.

FIG. 3 shows an example sequencing scheme of using a primary probe with a template nonhybridizing sequence. In this example, a template 301 is bound to a sequencing primer 302 and a primary probe 303 (e.g., a nucleic acid probe) having a template hybridizing sequence and a template nonhybridizing sequence. The template nonhybridizing sequence can initiate an amplification reaction to amplify a signal upon binding of one or more secondary probes. In this example, a branched DNA (bDNA) reaction is initiated via the template nonhybridizing sequence of the primary probe to amplify a detection signal. To initiate a bDNA reaction, the template nonhybridizing sequence can bind to a preamplifier 304 having a plurality of subsequences that can bind to a plurality of nucleic acid amplifiers 305. Each nucleic acid amplifier 305 can bind to one or more probes 306 having a detectable label 307 attached thereto. In the example shown in FIG. 3, the system is a 3×2 system where the preamplifier 304 contains binding sites for 3 amplifiers 305, and each amplifier has binding sites for two fluorescently labelled probes 306/307. In bDNA reactions of the disclosure, the number of amplifier binding sites on a preamplifier and/or the number of probe binding sites on an amplifier can vary. For example, a preamplifier can have binding sites for 1, 2, 3, 4, 5, 6, 7, 8, 9, 10, or more amplifiers. In some embodiments, an amplifier can have binding sites for 1, 2, 3, 4, 5, 6, 7, 8, 9, 10, or more probes.

In some embodiments, the bDNA amplification system can comprise a plurality of layers of preamplifiers, amplifiers, and probes. For example, a first preamplifier can comprise a plurality of binding sites to which a plurality of second preamplifiers bind. The second preamplifiers can comprise a plurality of binding sites to which a plurality of amplifiers bind. The amplifiers can then comprise a plurality of probes. An example of such an embodiment is a 2×2×2 system, wherein an initial preamplifier (a first preamplifier) contains binding sites for 2 secondary pre-amplifiers (a second preamplifier). The secondary preamplifiers contain 2 binding sites for 2 amplifiers. The amplifiers contain 2 sites for fluorescently labeled probes.

The sample used in the methods provided herein can be biological sample. The sample can be a cell or a tissue. The sample can be fixed. The sample can be permeabilized. The sample can be fixed and permeabilized. The sample can comprise a three-dimensional (3D) matrix. The 3D matrix can be a synthetic 3D matrix. The sample can be immobilized on a surface. In some cases, the nucleic acid molecule to be identified can be immobilized on a surface.

The signal provided herein can be various types of signals. The signal can be a fluorescent signal. The signal can comprise a plurality of fluorophores. The signal can be an optical signal. The signal can be an electrical signal or an electrochemical signal. The electrical signal can be a conductivity signal, impedance signal, or a charge signal. The signal can be removed or rendered undetectable.

The signal can be removed from the amplification product or from the nucleic acid molecule to be identified (e.g., a target nucleic acid molecule or template nucleic acid molecule). For example, the signal can be removed through removing the detectable labels discussed herein. In some cases, the detectable label can be quenched (e.g., by photobleaching) or physically removed. For another example, the signal can be removed through removing (e.g., disrupting or disassembling) the amplification product. The disruption or disassembly of the amplification product can comprise reversing the amplification reaction such that the amplification product becomes individual nucleic acid amplifiers. For yet another example, the signal can be removed through removing the amplification product without disassembling the amplification product. In such cases, the template nonhybridizing sequence attached to the amplification product may be removed from the template hybridizing sequence of the nucleic acid probe. Additionally or alternatively, the backbone of secondary amplification probes (e.g. secondary oligos) can include one or more cleavable linkages. Fragmenting an assembled nucleic acid structure (e.g. an HCR polymer or bDNA assembly) via cleavage of a cleavable linkage can allow for rapid washing or removal of an amplification product after detection. Cleavable linkages and cleavage mechanisms include, for example, any cleavable linkage or cleavage mechanism disclosed herein. In some embodiments, duplex regions of an assembled nucleic acid structure can be denatured to facilitate rapid removal from a sample by washing. Denaturation can be facilitated by exposing nucleic acid structures to, for example, heat, base treatment, formamide, and/or competitive hydrogen bonders.

In some cases, disassembly of the amplification product comprises a toehold mediated strand displacement reaction. In some embodiments, the amplification product comprises at least one additional single-stranded domain, known as a toe-hold, which is complementary to a strand of DNA referred to as the “invading strand.” The invading strand can be a single-stranded DNA molecule that acts as a strand-displacing oligonucleotide, which initiates a strand-displacement reaction by binding the single-stranded domain of the amplification product and then competitively displacing the template DNA strand through branch migration.

The sequencing primer provided herein can be ligated with the at least one nucleic acid probe.

The template hybridizing sequence can be removably (e.g., cleavably) attached to the template nonhybridizing sequence. The template hybridizing sequence can be cleavably attached to the template nonhybridizing sequence via a cleavable linker. The cleavable linker can be a photocleavable linker or a chemically cleavable linker. The template nonhybridizing sequence may be removed from the template hybridizing sequence to generate an extendable terminus on the template hybridizing sequence. In some cases, after removing the template nonhybridizing sequence, an additional nucleic acid probe having a template hybridizing sequence having a sequence that is complementary with the nucleic acid sequence and a template nonhybridizing sequence can be used to repeat the process of the methods described herein to identify additional nucleotides of the nucleic acid molecule. In some embodiments, additional nucleic acid probes can then repeatedly be hybridized and ligated in series along the nucleic acid molecule wherein after each ligation, one or more or all of the nucleotides of the hybridized and ligated oligonucleotide probe are identified and one or more or all of the complementary nucleotides in the nucleic acid molecule are identified.

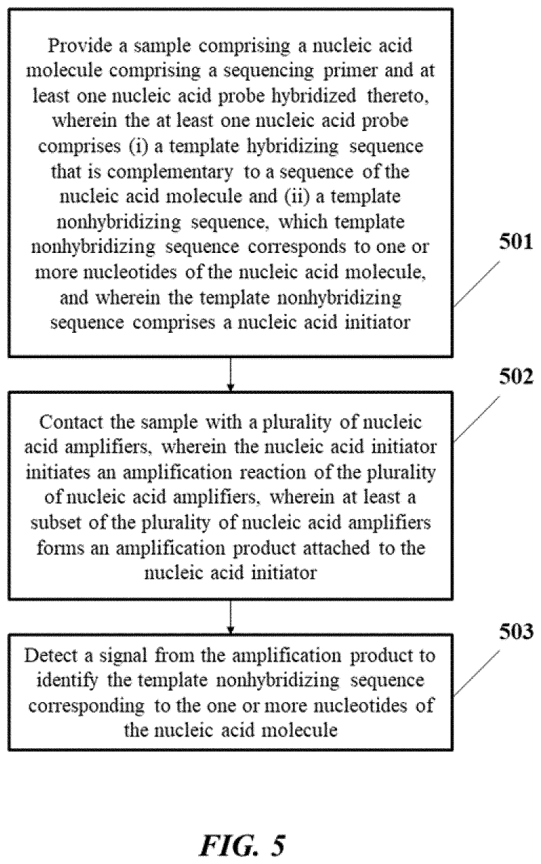

FIG. 5 shows an example method of sequencing provided in the present disclosure. In a first operation 501, a sample comprising a nucleic acid molecule is provided. The nucleic acid molecule hybridizes to a sequencing primer and at least one nucleic acid molecule. The at least one nucleic acid probe comprises (i) a template hybridizing sequence that is complementary to a sequence of the nucleic acid molecule and (ii) a template nonhybridizing sequence, which template nonhybridizing sequence corresponds to one or more nucleotides of the nucleic acid molecule. The template nonhybridizing sequence comprises a nucleic acid initiator. Next, in a second operation 502, the sample is contacted with a plurality of nucleic acid amplifiers. The nucleic acid initiator initiates an amplification reaction of the plurality of nucleic acid amplifiers. At least a subset of the plurality of nucleic acid amplifiers forms an amplification product attached to the nucleic acid initiator. Next, in a third operation, a signal is detected from the amplification product to identify the template nonhybridizing sequence corresponding to the one or more nucleotides of the nucleic acid molecule.

In some embodiments, a template nonhybridizing sequence can comprise multiple initiator motifs. Inclusion of multiple initiator motifs can allow for more than one base in a template hybridizing sequence to be represented by a nucleic acid probe. In some embodiments, a plurality of nucleic acid probes can be used simultaneously, wherein each probe contains a single initiator representing a single base identity of the template hybridizing region. Use of a plurality of nucleic acid probes simultaneously can allow multiple base positions of a template to be interrogated in parallel when a spatially-colocalized collection of clonal sequencing templates (e.g. a rolony, polymerase colony, etc.) has been generated from a template nucleic acid. After parallel interrogation of multiple base positions, the base positions can be read out in series. For example, a sub-population of nucleic acid probes with base 1 initiator motifs can be read in cycle 1, a sub-population of nucleic acid probes with base 2 initiator motifs can be read in cycle 2, and so on.

In some embodiments, the template non-hybridizing sequence motifs are functionally connected to the initiator by a secondary linker. Some of such compositions and methods are compatible with four orthogonal amplification systems, which can detect signals associated with four fluorescence colors. The methods generally include using a secondary linker motif to connect a template-non-hybridizing motif within a detection cycle with one of the initiator motifs. The results of each cycle are then detected using the fluorescent probes. These methods can be compatible with bDNA or HCR, including the methods described herein. Such compositions and methods can result in a reduction in the number of amplification reagents used within a sequencing system, including a reduction in the amount of costly fluorescence or cleavage moieties used.

In some embodiments, template non-hybridizing sequences may be duplexed or partially duplexed. Alternatively or additionally, a template non-hybridizing sequences detected in a first cycle can be hybridized to a first set of secondary linkers or initiators prior to or during the first cycle, which can render the template non-hybridizing sequence competent for amplification.

### Compositions for Detection

The present disclosure also provides a composition for identifying one or more nucleotides of a nucleic acid molecule in a sample. The composition can comprise the sample comprising the nucleic acid molecule comprising a sequencing primer and at least one nucleic acid probe hybridized thereto. The at least one nucleic acid probe can comprise (i) a template hybridizing sequence that is complementary to a sequence of the nucleic acid molecule and (ii) a template nonhybridizing sequence. The template nonhybridizing sequence can correspond to one or more nucleotides of the nucleic acid molecule. The template nonhybridizing sequence can comprise a nucleic acid initiator. The composition can further comprise a plurality of nucleic acid amplifiers.

In some cases, at least a subset of the plurality of nucleic acid amplifiers can form an amplification product attached to the nucleic acid initiator.

The present disclosure also provides kits for identifying one or more nucleotides of a nucleic acid molecule. A kit can comprise a sequencing primer. The kit can further comprise at least one nucleic acid probe. The at least one nucleic acid probe can comprise (i) a template hybridizing sequence that is complementary to a sequence of the nucleic acid molecule and (ii) a template nonhybridizing sequence. The template nonhybridizing sequence can correspond to the one or more nucleotides of the nucleic acid molecule. The template nonhybridizing sequence can comprise a nucleic acid initiator. The kit can further comprise a plurality of nucleic acid amplifiers. The kit can further comprise instructions that direct a user to use the methods provides herein. For example, the kit can comprise instructions that direct a user to (a) provide the nucleic acid molecule having hybridized thereto the sequencing primer and the at least one nucleic acid probe; (b) contact the nucleic acid molecule with the plurality of nucleic acid amplifiers such that the nucleic acid initiator of the template nonhybridizing sequence initiates an amplification reaction of at least a subset of the plurality of nucleic acid amplifiers to form an amplification product attached to the nucleic acid initiator; (c) detect a signal from the amplification product to identify one or more other nucleotides of the template nonhybridizing sequence, which one or more other nucleotides corresponds to the one or more nucleotides of the nucleic acid molecule; and (d) use at least the one or more other nucleotides identified in (c) to identify the one or more nucleotides of the nucleic acid molecule.

### Target Nucleic Acid Molecules

Target nucleic acid molecules, also referred to as template nucleic acid molecules, to be sequenced according to the methods described herein can be prepared in a variety of ways.

The target nucleic acid molecules can be single stranded nucleic acid molecules. The length of the target nucleic acid molecule can vary. The length of the target nucleic acid molecule can be between about 1 nucleotide to about 3,000,000 nucleotides in length, between about 1 nucleotide to about 2,500,000 nucleotides in length, between about 1 nucleotide to about 2,000,000 nucleotides in length, between about 1 nucleotide to about 1,500,000 nucleotides in length, between about 1 nucleotide to about 1,000,000 nucleotides in length, between about 1 nucleotide to about 500,000 nucleotides in length, between about 1 nucleotide to about 250,000 nucleotides in length, between about 1 nucleotide to about 200,000 nucleotides in length or between about 1 nucleotide to about 150,000 nucleotides in length. Example target nucleic acid molecule can be between about 1 nucleotide to about 100,000 nucleotides in length, between about 1 nucleotide to about 10,000 nucleotides in length, between about 1 nucleotide to about 5,000 nucleotides in length, between about 4 nucleotides to about 2,000 nucleotides in length, between about 6 nucleotides to about 2,000 nucleotides in length, between about 10 nucleotides to about 1,000 nucleotides in length, between about 20 nucleotides to about 100 nucleotides in length, and any range or value in between whether overlapping or not.

A target for sequencing can be prepared from several linear or circular sources of polynucleotides, such as dsDNA, ssDNA, cDNA, RNA and synthesized or naturally occurring polynucleotides.

An example template can be a synthesized polynucleotide of the form 5′-PO₄-GTT CCT CAT TCT CTG AAG ANN NNN NNN NNN NNN NNN NNN NNN NNN NNN NNN NNN NAC TTC AGC TGC CCC GG-3′-OH (SEQ ID NO: 1), where the N portion represents a ssDNA template to be identified, GTT CCT CAT TCT CTG AAG A (SEQ ID NO: 2) and AC TTC AGC TGC CCC GG (SEQ ID NO: 3) represent adapters that can be used as a sequencing primer hybridization site. Sequencing can be accomplished in either the 5′ to 3′ direction or the 3′ to 5′ direction or both directions simultaneously. Multiple copies of the template nucleic acid can be prepared. The ssDNA template can be circularized using ssDNA Circligase II or other ssDNA ligase such as Circligase I, or by template-directed ligation using a combination of a dsDNA ligase (e.g., T3, T4, T7 and other dsDNA ligases) with a bridge oligo (5′-ATGAGGAACCCGGGGCAG-3′-PO₄) (SEQ ID NO: 4).

10 pmol of ssDNA template can be circularized using Circligase II, according to the manufacturer's recommendation. Following the circularization, 20 units of Exonuclease I and 100 units of Exonuclease III can be added to the reaction to digest any remaining linear template. Next, rolling circle amplification (RCA) can be performed on the circular ssDNA template using a DNA polymerase with high processivity, strong displacement activity and low error rate. 1 pmol of the circularized template can be used with 20 units of phi29 DNA polymerase. Additionally, dNTP (e.g., 1 mM) and a RCA primer (e.g., 1 pmol) may be used. An example RCA primer may have the form 5′-AATGAGGAACCCGGGGCA*G*C (SEQ ID NO: 5), where the * represents a phosphorothioate bond thereby indicating that the last 3′ nucleotide bears a phosphorothioate bond, making the RCA less susceptible to phi29 3′→5′ exonuclease activity. However, an example RCA primer may not include such phosphorothioate bonds, especially if the polymerase used does not have 3′→5′ exonuclease activity. Alternatively, an example RCA primer may have phosphorothioate bonds on the 5′ side of the RCA primer such as 5′-A*A*TGAGGAACCCGGGGCAGC (SEQ ID NO: 6). An annealing reaction may be performed before adding the phi29 (95° C. for 1 min, then 2 min cool down to 4° C.), to increase the RCA efficiency. Then the reaction can be incubated at 30° C. for an hour (incubation periods between 15 min to 6 hours may also be used). Other temperatures can be used, since phi29 can be active between 4° C. and 40° C. (with 90% diminished activity). Then, the reaction can be cooled to 4° C. and the RCA products (referred to as rolonies) can be recovered in cold PBS and can be stored at 4° C. until needed. Rolling circle amplification products prepared this way may be stable for several months and can be used as template for assaying sequencing techniques.

A template can also be prepared using dsDNA from a biological source. The genomic DNA may first be extracted using one of the several extraction kits commercially available for that purpose. Then, the dsDNA can be fragmented to random lengths or specific lengths, using mechanical (e.g., using a focused electroacoustic device, a nebulizer, sonication or a vortex) or enzymatic (e.g., fragmentase) fragmentation methods. While, it may be practical to keep the fragments size between 100 and 1000 nucleotides, other sizes can be used. In certain instances, the target may be an entire strand of a genomic DNA or a portion or fragment thereof.

The ends of the fragmented dsDNA can be repaired and phosphorylated in one operation using a mix of T4 DNA polymerase and T4 Polynucleotide Kinase, according to the manufacturer instructions. Other DNA polymerase with 3′→5′ exonuclease activity and low or no strand displacement activity can be used. Adapters composed of dsDNA oligonucleotides can be added to the dsDNA using a DNA ligase, such as, for example, T3 or T4 DNA ligase. The reaction can be performed at room temperature for 20 min according to the manufacturer instructions. The adapters can be in the form Ad1 5′-GTTCCTCATTCTCTGAAGA (SEQ ID NO: 7), Ad2 5′-TCTTCAGAGAATGAG (SEQ ID NO: 8), Ad3 5′-CCGGGGCAGCTGAAGT (SEQ ID NO: 9), and Ad4 5′-ACTTCAGCTGCC (SEQ ID NO: 10), where Ad1-Ad2 are annealed together and Ad3-Ad4 anneal together, before being ligated. After ligation, the 5′ overhang ends can be filled-in using a DNA polymerase with, such as Bst DNA polymerase large fragment. Next, limited PCR (e.g., at least 5, 6, 7, 8, 9, or 10 cycles) can be performed to generate multiple copies using PCR primer in the form 5′-PO₄-GTTCCTCATTCTCTGAAGA (SEQ ID NO: 7) and 5′-Biotin-CCGGGGCAGCTGAAGT (SEQ ID NO: 9). The 5′ biotin can then be attached to one end of the dsDNA to streptavidin coated magnetic beads, allowing the other end to be recovered by performing the Circligase II reaction, as described above, with the exception that the template is attached to the beads. This can be performed by incubating the reaction at 65° C. for 2 h, which can allow the DNA strand with 5′-PO₄ to be de-anneal and be circularized. After exonuclease digest, the circular ssDNA template is now ready for rolling circle amplification (RCA) as discussed above.

Adapters can also be in the form Ad5 5′-GAAGTCTTCTTACTCCTTGGGCCCCGTCAGACTTC (SEQ ID NO: 11) and Ad6 5′-GTTCCGAGATTTCCTCCGTTGTTGTTAATCGGAAC (SEQ ID NO: 12), where Ad5 and Ad6 each form hairpin structures to be ligated on each side of the dsDNA, virtually creating a circular ssDNA product ready for RCA. A pull-down assay can be used to select templates bearing one of each hairpin and not two of the same. In this case, an oligonucleotide complementary to one loop in the form 5′-Biotin-TAACAACAACGGAGGAAA-C3sp (SEQ ID NO: 13) can be bound to streptavidin coated magnetic beads. Next RCA can be performed using a RCA primer (5′-ACGGGGCCCAAGGAGTA*A*G) (SEQ ID NO: 15), as described above.

Other amplification methods can be used. The amplification method can be isothermal. The amplification can be in situ amplification methods such as the methods described in U.S. Pat. No. 6,432,360, which is incorporated herein by reference. Examples of amplification methods include, but are not limited to, polymerase chain reaction (PCR), anchor PCR, RACE PCR, ligation chain reaction (LCR), self-sustained sequence replication, transcriptional amplification system, Q-Beta Replicase, recursive PCR, and the amplification methods described in U.S. Pat. Nos. 6,391,544, 6,365,375, 6,294,323, 6,261,797, 6,124,090 and 5,612,199, each of which is incorporated herein by reference.

### Support

In some embodiments, one or more template nucleic acid molecules described herein can be immobilized on a support (e.g., a solid and/or semi-solid support). In certain aspects, a target nucleic acid molecule can be attached to a support using one or more of the phosphoramidite linkers. Suitable supports include, but are not limited to, slides, beads, chips, particles, strands, gels, sheets, tubing, spheres, containers, capillaries, pads, slices, films, plates and the like. In various embodiments, a solid support may be biological, nonbiological, organic, inorganic, or any combination thereof. When using a support that is substantially planar, the support may be physically separated into regions, for example, with trenches, grooves, wells, or chemical barriers (e.g., hydrophobic coatings, etc.).

In certain embodiments, a support can be a microarray. As used herein, the term “microarray” refers to a type of assay that comprises a solid phase support having a substantially planar surface on which there is an array of spatially defined non-overlapping regions or sites that each contain an immobilized hybridization probe. “Substantially planar” means that features or objects of interest, such as probe sites, on a surface may occupy a volume that extends above or below a surface and whose dimensions are small relative to the dimensions of the surface. For example, beads disposed on the face of a fiber optic bundle create a substantially planar surface of probe sites, or oligonucleotides disposed or synthesized on a porous planar substrate creates a substantially planar surface. Spatially defined sites may additionally be “addressable” in that its location and the identity of the immobilized probe at that location are known or determinable.

Oligonucleotides immobilized on microarrays include nucleic acids that are generated in or from an assay reaction. Oligonucleotides or polynucleotides on microarrays can be single stranded and can be covalently attached to the solid phase support, usually by a 5′-end or a 3′-end. In certain embodiments, probes can be immobilized via one or more of the cleavable linkers described herein. The density of non-overlapping regions containing nucleic acids in a microarray may be greater than 100 per cm², or greater than 1000 per cm².

### Sequencing Primers

The methods provided herein comprise hybridizing a sequencing primer to a target nucleic acid molecule. The sequence primer can bind to a known binding region of the target nucleic acid molecule and facilitating ligation of a nucleic acid probe of the present disclosure. Sequencing primers may be designed with the aid of a computer program such as, for example, DNAWorks, or Gene2Oligo. The binding region can vary in length but it can be long enough to hybridize the sequencing primer. Target nucleic acid molecules may have multiple different binding regions thereby allowing different sections of the target nucleic acid molecules to be sequenced. Sequencing primers can be selected to form highly stable duplexes so that they remain hybridized during successive cycles of ligation. Sequencing primers can be selected such that ligation can proceed in either the 5′ to 3′ direction or the 3′ to 5′ direction or both. Sequencing primers may contain modified nucleotides or bonds to enhance their hybridization efficiency, or improve their stability, or prevent extension from a one terminus or the other.

For the purpose of identifying several template nucleotide sequences in parallel, the target nucleic acid molecules can be diluted in a buffer (e.g., PBS buffer pH 7.4) and either bound to a patterned or non-patterned substrate utilizing various attachment methods, such as biotin-streptavidin, azide-alkyne (e.g., click chemistry), NETS-ester or silanization (e.g., aldehyde-, epoxy-, amino-silane).

In some cases, the target nucleic acid molecules may be rolonies. The rolonies can be attached to a patterned surface, such as a SiO₂ solid surface, treated with 1% aminosilane (v/v) and let to interact for a period of time (e.g., between 5 minutes to 2 hours). Any unbound target nucleic acid molecules can be washed away.

Sequencing primers can be prepared which can hybridize to a known sequence of the target nucleic acid molecule. Alternatively, during template preparation, adapters with a known nucleic acid sequence are added to the unknown nucleic acid sequence of a target nucleic acid molecule by way of ligation, amplification, transposition or recombination. In some cases, sequencing primers having a certain level of degeneracy can be used to hybridize to certain positions along the target nucleic acid molecule. Primer degeneracy can be used to allow primers to hybridize semi-randomly along the target nucleic acid molecule. Primer degeneracy may be selected based on statistical methods to facilitate primers hybridizing at certain intervals along the length of the target nucleic acid molecule. Primers can be designed having a certain degeneracy which facilitates binding every N bases, such as every 100 bases, every 200 bases, every 300 bases, every 400 bases, every 500 bases, every 1,000 bases, every 2,000 bases, every 5,000 bases, every 10,000 bases, every 50,000 bases, every 100,000 bases or more. The binding of the primers along the length of the target nucleic acid molecule can be based on the design of the primers and the statistical likelihood that a primer design can bind about every N bases along the length of the target nucleic acid molecule. Since the sequencing primer can be extended by ligation, the terminal group of the sequencing primer can be synthesized to be ready to be covalently joined to the nucleic acid probe by the DNA ligase. If the ligation occurs between the 5′ end of the sequencing primer and the 3′ end of the nucleic acid probe, a phosphate group (5′-PO₄) can be present on the sequencing primer while a hydroxyl group (3′-OH) on the nucleic acid probe, and vice-versa.

### Nucleic Acid Probes

The methods provided herein comprising hybridizing a nucleic acid probe to a target nucleic acid molecule. The nucleic acid probe can have at least about 2, 5, 10, 20, 25, 30, 35, 40, 45, 50, 55, 60, 65, 70, 75, 80, 85, 90, 95, 100, or more nucleotides. In some cases, the nucleic acid probe can hybridize to the single stranded target nucleic acid molecule. In some cases, the nucleic acid probe can hybridize to the single stranded target nucleic acid molecule and be ligated to a sequencing primer. Nucleic acid probes may be designed with the aid of a computer program such as, for example, DNAWorks, or Gene2Oligo.

The nucleic acid probe can comprise a template hybridizing sequence and a template nonhybridizing sequence. The template nonhybridizing sequence can be removeable (e.g., cleavably) attached to the template hybridizing sequence. In some cases, the template nonhybridizing sequence can be linked to the template hybridizing sequence via a cleavable linkage. The template nonhybridizing sequence can be removed (e.g., cleaved) from the template hybridizing sequence to generate an extendable terminus, which may be used for another round of detection by hybridizing an additional nucleic acid probe.

### Cleavable Linkages

Cleavable linkages include, but are not limited to, chemically scissile internucleosidic linkages, which may be cleaved by treating them with chemicals or subjecting them to oxidizing or reducing environments. An example of such cleavable linkage includes phosphorothioate or phosphorothiolate which can be cleaved by various metal ions such as solutions of silver nitrate. Another example of such cleavable linkage includes phosphoroamidate, which can be cleaved in acidic conditions such as solutions including acetic acid. A suitable chemical that can cleave a linkage includes a chemical that can cleave a bridged-phosphorothioate linkage and can remove a phosphoramidite linker from a nucleotide and/or oligonucleotide, leaving a free phosphate group on the nucleotide and/or oligonucleotide at the cleavage site. Suitable chemicals include, but are not limited to, AgNO₃, AgCH₃COO, AgBrO₃, Ag₂SO₄, or any compound that delivers Ag²⁺, HgCl₂, I₂, Br₂, I⁻, Br⁻ and the like.

Cleavable linkages also include those that can be cleaved by nucleases. Examples of nucleases include restriction endonucleases such as Type I, Type II, Type III and Type IV, endonucleases such as endonucleases I-VIII, ribonucleases and other nucleases such as enzymes with AP endonuclease activity, enzymes with AP lyase activity and enzymes with glycosylase activity such as uracil DNA glycosylase.

Cleavable linkages also include those capable of being cleaved by light of a certain wavelength. Examples of such cleavable linkages can be photolabile or photocleavable linkages such as photocleavable biotin derivatives. In some cases, the photocleavable linkages can be cleaved by UV illumination between wavelengths of about 275 to about 375 nm for a period of a few seconds to 30 minutes, such as about one minute. Example wavelengths include between about 300 nm to about 350 nm.

Certain nucleotides, such as dGTP, dCTP and dTTP can be reacted before being incorporated for use as a cleavable linkage, making them specifically sensitive to further cleavage by nucleases or chemicals. In some cases, one or multiple deoxyguanosines in a given template nonhybridizing sequence can be oxidized to 8-oxo-deoxyguanosine by 2-nitropropane, before being added to the sequencing reaction, and subsequently cleaved using an 8-oxoguanine DNA glycosylase (e.g., Fpg, hOGG1). Similarly, deoxycytosines can be pre-reacted to form 5-hydroxycytosine, using bisulfite or nitrous acid, which can then be processed by certain DNA-glycosylase, such as hNEIL1. Other examples of nucleotides that can be cleaved include uracil, deoxyuridine, inosine and deoxyinosine.

The cleavable linkage may be cleaved in a two-operation method such as by a first operation that modifies a nucleotide of the cleavable linkage making it more susceptible to cleavage and then a second operation where the nucleotide is cleaved. Such systems include the USER system which can be a combination of UDG and Endonuclease VIII, although other endonucleases may be used. Enzymes UDG and endonuclease are commercially available. In some cases, a nucleotide linking the template nonhybridizing sequence and the template hybridizing sequence can be modified to be a cleavable nucleotide, where a feature of the nucleotide has been modified, such as a bond, so as to facilitate cleavage. Examples include an abasic base, an apyrimidic base, an apurinic base, phosphorothioate, phosphorothiolate and oxidized bases such as deoxyguanosines which can be oxidized to 8-oxo-deoxyguanosine.

Internucleotide bonds may be cleaved by chemical, thermal, or light-based cleavage. Examples of chemically cleavable internucleotide linkages for use in the methods described herein include, but are not limited to, β-cyano ether, 5′-deoxy-5′-aminocarbamate, 3′-deoxy-3′-aminocarbamate, urea, 2′-cyano-3′,5′-phosphodiester, 3′-(S)-phosphorothioate, 5′-(S)-phosphorothioate, 3′-(N)-phosphoramidate, 5′-(N)-phosphoramidate, α-amino amide, vicinal diol, ribonucleoside insertion, 2′-amino-3′,5′-phosphodiester, allylic sulfoxide, ester, silyl ether, dithioacetal, 5′-thio-furmal, α-hydroxy-methyl-phosphonic bisamide, acetal, 3′-thio-furmal, methylphosphonate and phosphotriester. Internucleoside silyl groups such as trialkylsilyl ether and dialkoxysilane can be cleaved by treatment with fluoride ion. Base-cleavable sites include β-cyano ether, 5′-deoxy-5′-aminocarbamate, 3′-deoxy-3′-aminocarbamate, urea, 2′-cyano-3′,5′-phosphodiester, 2′-amino-3′,5′-phosphodiester, ester and ribose. Thio-containing internucleotide bonds such as 3′-(S)-phosphorothioate and 5′-(S)-phosphorothioate can be cleaved by treatment with silver nitrate or mercuric chloride. Acid cleavable sites include 3′-(N)-phosphoramidate, 5′-(N)-phosphoramidate, dithioacetal, acetal and phosphonic bisamide. An α-aminoamide internucleoside bond can be cleavable by treatment with isothiocyanate, and titanium may be used to cleave a 2′-amino-3′,5′-phosphodiester-O-ortho-benzyl internucleoside bond. Vicinal diol linkages can be cleavable by treatment with periodate. Thermally cleavable groups include allylic sulfoxide and cyclohexene while photolabile linkages include nitrobenzyl ether and thymidine dimer. Methods synthesizing and cleaving nucleic acids containing chemically cleavable, thermally cleavable, and photolabile groups are described for example, in U.S. Pat. No. 5,700,642.

The template nonhybridizing sequence can comprise an initiator (e.g., an initiator sequence). The initiator can be used to initiate an amplification reaction to generate an amplification product. The amplification product can comprise a signal for detection. The amplification reaction can be a hybridization chain reaction (HCR), where HCR monomers self-assemble (e.g., polymerize) to generate a HCR polymer. The amplification reaction can be a branched nucleic acid amplification to generate a branched nucleic acid structure bearing a signal strong enough for detection.

### Hybridization Chain Reaction

The amplification reaction described herein can be a hybridization chain reaction (HCR), which is a method for the triggered hybridization of nucleic acid molecules starting from nucleic acid monomers (e.g., metastable monomer hairpins or other metastable nucleic acid structures). Methods and compositions of HCR system are described in, for example, U.S. Pat. No. 8,124,751B2, which is incorporated by reference herein in its entirety. HCR may not require any enzymes and can operate isothermally. HCR amplifies the signal by increasing the number of detectable labels, such as fluorophores, localized to the initiator. The initiator can be said to be information encoding to the extent that initiators can be designed to be associated with a particular target molecule within a sample including a plurality of target molecules. In the case of sequencing methods described herein, the initiator of the template nonhybridizing sequence can be associated with one or more particular nucleotides to be detected of the template hybridizing sequence.

In some cases, two or more metastable monomer hairpins can be used. In some cases, two metastable monomer hairpins (e.g., two species of metastable monomer hairpins) can be used, where the two metastable monomer hairpins have different sequences. The hairpins can comprise loops that are protected by long stems. The loops can thus be resistant to invasion by complementary single-stranded nucleic acids. This stability may allow the storage of potential energy in the loops. Potential energy can be released when a triggered conformational change allows the single-stranded bases in the loops to hybridize with a complementary strand, for example, in a second hairpin monomer. When two species of monomer hairpins are used, they can hybridize in an alternate pattern to form a HCR polymer.

Each monomer can be caught in a kinetic trap, preventing the system from rapidly equilibrating. That is, pairs of monomers may be unable to hybridize with each other in the absence of an initiator. Introduction of an initiator strand can cause the monomers to undergo a chain reaction of hybridization events to form a nicked helix (e.g., FIG. 2).

Each monomer can comprise at least one region that is complementary to at least one other monomer being used for the HCR reaction. A first monomer in a monomer pair can comprise an initiator complement region that is complementary to a portion of an initiator molecule or sequence. The initiator complement region can be a sticky end. Binding of the initiator to the initiator complement region can start an HCR. In addition, the second monomer can comprise a propagation region that is able to hybridize to the initiator complement region of another monomer or another copy of the first monomer, to continue the HCR initiated by the initiator. The propagation region may be, for example, the loop region of a hairpin monomer. In some cases, the propagation region on the second monomer may be identical to the portion of the initiator that is complementary to the initiator complement region of the first monomer. The propagation region on the second monomer may be available to interact with the initiator complement region of the first monomer when an HCR has been started by the initiator. That is, the propagation region may become available to hybridize to the initiator complement region of another monomer when one copy of the first monomer has already hybridized to a second monomer.

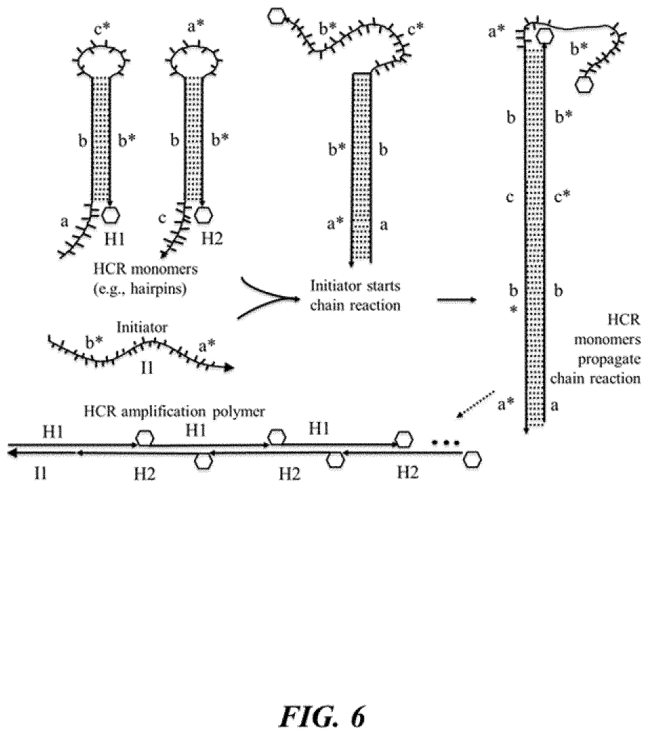

FIG. 6 depicts an example HCR mechanism. Metastable fluorescent hairpins self-assemble into fluorescent amplification polymers upon detection of a cognate initiator. Initiator I1, comprised of single-stranded segments “b*-a*”, nucleates with hairpin H1 via base-pairing to single-stranded toehold “a” of H1, mediating a branch migration that opens the hairpin to form complex I1.H1 containing single-stranded segment “c*-b*”. This complex nucleates with hairpin H2 using, for example, base-pairing to single-stranded toehold “c”, mediating a branch migration that opens the hairpin to form complex I1.H1.H2 containing single-stranded segment “b*-a*”. Thus, the initiator sequence is regenerated, providing the basis for a chain reaction of alternating H1 and H2 polymerization. Stars denote fluorophores.

The length of the loop, stem and sticky ends of the monomers can be adjusted, for example to ensure kinetic stability in particular reaction conditions and to adjust the rate of polymerization in the presence of initiator. In some cases, the length of the sticky ends can be the same as the length of the loops. In some cases, the sticky ends can be longer or shorter than the loops. However, if the loops are longer than the sticky ends, the loops can comprise a region that is complementary to the sticky end of a monomer. In some cases, the length of the loops can be short relative to the stems. For example, the stems may be two or three times as long as the loops. The loop regions can be between about 1 and about 100 nucleotides, and in some cases, between about 3 and about 30 nucleotides and in some cases, between about 4 and about 7 nucleotides. In some cases, the loops and sticky ends of a pair of hairpin monomers can be about 6 nucleotides in length and the stems can be about 18 nucleotides long.

Reaction conditions can be selected such that hybridization is able to occur, both between the initiator and the sticky end of a first monomer, and between the complementary regions of the monomers themselves. The reaction temperature may not need to be changed to facilitate the HCR. That is, the HCR reactions are isothermic. They may not require the presence of any enzymes.

Methods and compositions provided herein use HCR to amplify a signal for detecting (e.g., imaging) one or more nucleotides of a target nucleic acid molecule. The advantages of HCR include, without limitation, the ability to rapidly amplify a signal based on a small amount of target nucleic acid molecules present and the ability to image a diversity of target nucleic acid molecules in the same sample. Self-quenching HCR monomers can be labeled with fluorophore/quencher pairs that become separated during self-assembly into tethered amplification polymers. This active background suppression may be useful in the situations where unused amplification components cannot be washed away before detection (e.g., imaging).

The HCRs described herein can be programmable HCRs as described in the U.S. patent application Ser. No. 16/170,751, which is incorporated by reference herein in its entirety. For example, the associate between the initiator and the HCR polymer can be reversed such that the HCR polymer may be removed after detection. For another example, the associate between the HCR polymer and the signal (e.g., a plurality of detectable labels) can be reversed such that the signal can be removed after detection. Various methods can be used to achieve the reversibility of the HCRs. In some cases, the HCR monomers may contain functional groups for programmable disassembly or degradation of the polymer. In some cases, the functional groups can comprise toehold strand displacement sequences such that the HCR monomers can be displaced to disassemble the polymer by introducing nucleic acid strands to initiate a strand displacement reaction from the toehold strand displacement sequences. In some cases, the functional groups comprise chemically labile, enzymatically labile, or photolabile chemical groups. The cleavable linkages described herein can be used in designing HCR monomers. Modified HCR monomers comprising enzymatic, chemical, or photolabile groups between the HCR monomer backbone and the detectable labels may be used such that the detectable labels can be removed by chemical, enzymatic, or light treatments. Probes bearing detectable labels capable of labeling a HCR polymer may be used, where the probes comprise additional sequence for toehold strand displacement such that the probes can be removed from the HCR polymer by disrupting the hybridization between the probes and the HCR polymer. In some cases, cleavable linkages such as enzymatic, chemical, or photolabile groups may be used between the HCR polymer backbone and the detectable labels such that the detectable labels can be removed by chemical, enzymatic, or light treatments.

### Branched Nucleic Acid Amplification

The amplification reaction described herein can be a branched nucleic acid amplification, e.g., a branched DNA (bDNA) amplification. The branched nucleic acid amplification described herein can be an in-situ hybridization. The bDNA-based methods can effectively provide for amplification of a signal that may otherwise be undetectable. In bDNA amplification, the template nonhybridizing sequence of the nucleic acid probe can bind to an additional probe, referred to as a preamplifier in the present disclosure. The preamplifier can hybridize to a plurality of nucleic acid amplifiers (e.g., FIG. 3, the “L” shaped nucleic acid strand 305). The nucleic acid amplifier, in a bDNA-based reaction, can comprise a first portion that can hybridize with a portion of the preamplifier and a second portion that is not hybridizable to the preamplifier. Probes having detectable labels can be introduced to hybridize with the second portion of the nucleic acid amplifier that is not hybridizable to the preamplifier such that the amplification product can be detected.

Hybridization between components of the bDNA-based amplification system may need certain conditions. The time, temperature and pH conditions used to accomplish hybridization depend on the size of the oligonucleotide probe to be hybridized, the degree of complementarity between the oligonucleotide probe and the target, and the presence of other materials in the hybridization reaction admixture. Examples of hybridization conditions include the use of solutions buffered to a pH from about 7 to about 8.5 and temperatures from about 30° C. to about 55° C. (or from about 37° C. to about 55° C.) for a time period of from about 1 second to about 1 day (e.g., from about 15 minutes to about 16 hours or from about 15 minutes to about 3 hours). Any buffer that is compatible (e.g., chemically inert) with respect to the probes and other components, yet still allows for hybridization between complementary base pairs, can be used. An example buffer can comprise 3×SSC, 50% formamide, 10% dextran sulfate (MW 500,000), 0.2% casein, 10 μg/ml poly A, 100 μg/ml denatured salmon sperm DNA wherein 1×SSC is 0.15 M sodium chloride and 0.015 M sodium citrate. Another example buffer can comprise 5×SSC, 0.1 to 0.3% sodium dodecyl sulfate, 10% dextran sulfate, 1 mM ZnCl₂, and 10 mM MgCl₂.

### Hybridization and Ligation of Nucleic Acid Probes

The methods provided herein comprise hybridizing a nucleic acid probe to a target nucleic acid molecule and ligating the nucleic acid probe to a sequencing primer. Hybridization conditions can include salt concentrations of less than about 1 M, less than about 500 mM or less than about 200 mM. Hybridization temperatures can be as low as 5° C., but can be greater than 22° C., or greater than about 30° C., and in some cases in excess of about 37° C. Hybridizations may be performed under stringent conditions. Stringent conditions may be sequence-dependent and may be different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions can be selected to be about 5° C. lower than the Tₘ for the specific sequence at s defined ionic strength and pH. Examples of stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM Na phosphate, 5 mM EDTA, pH 7.4) and a temperature of 25-30° C. may be suitable for allele-specific probe hybridizations.

Ligation can be accomplished either enzymatically or chemically. Ligation can comprise forming a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations can be carried out enzymatically to form a phosphodiester linkage between a 5′ carbon of a terminal nucleotide of one oligonucleotide with 3′ carbon of another oligonucleotide.

Enzymatic ligation can utilize a ligase. Examples of ligases include, but are not limited to, T4 DNA ligase, T7 DNA ligase, E. coli DNA ligase, Taq ligase, Pfu ligase and the like. If ligation is not 100% efficient, it may be useful to cap extended duplexes that fail to undergo ligation so that they cannot participate in further ligation. Capping can be performed, for example, by removing the 5′phosphate (5′PO) using an alkaline phosphatase. For example, following ligation of the nucleic acid probes for sequencing, unreacted 5′PO can be removed by adding an alkaline phosphatase in solution, such as 10 units of calf intestinal alkaline phosphatase in 100 μL of its reaction buffer. The reaction can be incubated for 15 minutes at room temperature. Other alkaline phosphatases may be suitable. Capping can also be done by using a polymerase, deficient in exonuclease activity, to add a terminal nucleotide in the 5′→3′ direction (so capping the 3′ end of a primer). Terminal nucleotide may vary but examples of terminal nucleotide used include dideoxynucleotides (ddNTP) and acyclonucleotides (acyNTP). A nontemplated nucleotide can also be used as a terminal nucleotide. Capping by polymerase extension may be performed as described to amplify a polynucleotide sequence using DNA polymerases, except that dNTP used in the reaction may be substituted by terminal NTP (e.g., ddNTP), which can prevent the DNA polymerase or Terminal Transferase (TdT) of adding more than one nucleotide. For example, following ligation of the nucleic acid probes for sequencing, a capping mix can be added, which comprises 1 mM of ddNTP and 20 units of Terminal Transferase in 100 μL of its reaction buffer. The reaction can be incubated for 15 minutes at room temperature. Alternatively, capping can be done by ligating an oligonucleotide, e.g., between 6-9 nucleotides long, with a capped end. The cap can be in the form of 5′hydroxyl (5′OH), instead of 5′PO, and oppositely 3′PO instead of 3′OH, a terminal NTP (ddNTP, inverted ddNTP, acyNTP) or an oligo with a terminal carbon spacer (e.g., C3 spacer). This method may work as well for capping the 5′ end or the 3′ end of the polynucleotide sequence to be capped. Capping by ligation can be performed as described for ligating a nucleic acid probe. For example, following ligation of the nucleic acid probes for sequencing, a capping mix can be added, which comprises 1 μM of a 5′- or 3′-capped oligonucleotides added to the ligation buffer with 1200 units of T4 DNA ligase, per 100 μL reaction volume. The reaction can be incubated for 15 minutes at room temperature.

A set of nucleic acid probes can be utilized to hybridize to the ssDNA template and covalently linked to the sequencing primer by a DNA ligase. Nucleic acid probes can be prepared in ligation buffer (e.g., with final concentration of the probes at 1 μM) and ligated using 6000 units of T3 DNA ligase or 1200 units of T4 DNA ligase per 100 μL reaction volume. The reaction may be allowed to incubate at room temperature for a few minutes to several hours (e.g., between 5 minutes to 2 hours, at a temperature between 15° C. and 35° C.). Then the enzymes and any unligated nucleic acid probes can be washed away with a buffer (e.g., 10 mM Tris-HCl pH 7.5, 50 mM KCl, 2 mM EDTA pH 8.0, 0.01% Triton X-100 (v/v)).

### Signal

The signal provided herein can be various types of signals. The signal can be a fluorescent signal. The signal can comprise a plurality of fluorophores. The signal can be an optical signal. The signal can be an electrical signal or an electrochemical signal. The electrical signal can be a conductivity signal, impedance signal, or a charge signal. The signal can be removed or rendered undetectable.

The signal can comprise a detectable label or a plurality of detectable labels. The detectable label can be an optical label, e.g., a fluorophore.

Examples of detectable labels include various radioactive moieties, enzymes, prosthetic groups, fluorescent markers, luminescent markers, bioluminescent markers, metal particles, protein-protein binding pairs, protein-antibody binding pairs and the like. Examples of fluorescent moieties include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, cyanines, dansyl chloride, phycocyanin, phycoerythrin and the like. Examples of bioluminescent markers include, but are not limited to, luciferase (e.g., bacterial, firefly, click beetle and the like), luciferin, aequorin and the like. Examples of enzyme systems having visually detectable signals include, but are not limited to, galactosidases, glucorinidases, phosphatases, peroxidases, cholinesterases and the like. Identifiable markers also include radioactive compounds such as ¹²⁵I, ³⁵S, ¹⁴C or ³H. Identifiable markers are commercially available from a variety of sources.

The detectable label can be incorporated into the nucleic acid amplifier. Commercially available fluorescent nucleotide analogues readily incorporated into nucleotide and/or oligonucleotide sequences include, but are not limited to, Cy3-dCTP, Cy3-dUTP, Cy5-dCTP, Cy5-dUTP, fluorescein-12-dUTP, tetra-methylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHODAMINE GREEN™-5-UTP, ALEXA FLUOR™ 488-5-UTP, LEXA FLUOR™ 546-14-UTP and the like. Alternatively, the above fluorophores and those mentioned herein may be added during oligonucleotide synthesis using for example phosphoramidite or NHS chemistry. 2-Aminopurine is a fluorescent base that can be incorporated directly in the oligonucleotide sequence during its synthesis. A nucleic acid may also be stained, a priori, with an intercalating dye, such as, for example, DAPI, YOYO-1, ethidium bromide, cyanine dyes (e.g., SYBR Green) and the like.

The detectable label can be attached to the nucleic acid amplifier. Other fluorophores available for post-synthetic attachment include, but are not limited to, ALEXA FLUOR™ 350, ALEXA FLUOR™ 405, ALEXA FLUOR™ 430, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BOD-IPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, Pacific Orange, rhodamine 6G, rhodamine green, rhodamine red, tetramethyl rhodamine, Texas Red, Cy2, Cy3, Cy3.5, Cy5, Cy5.5, Cy7 and the like. FRET tandem fluorophores may also be used, including, but not limited to, PerCP-Cy5.5, PE-Cy5, PE-Cy5.5, PE-Cy7, PE-Texas Red, APC-Cy7, PE-Alexa dyes (e.g., 610, 647, 680), APC-Alexa dyes and the like.

Metallic silver or gold particles may be used to enhance signal from fluorescently labeled nucleotide and/or oligonucleotide sequences.

### Biological Sample

A biological sample may be provided in the methods, systems and compositions described herein. The biological sample can comprise the nucleic acid molecule to be detected (e.g., sequenced) using the methods described herein. The nucleic acid molecule can be detected in situ within the biological sample. The biological sample can comprise a three-dimensional (3D) matrix, for example, a 3D hydrogel matrix.

In some aspects, a biological sample may be fixed in the presence of a matrix-forming materials, for example, hydrogel subunits. By “fixing” the biological sample, it is meant exposing the biological sample, e.g., cells or tissues, to a fixation agent such that the cellular components become crosslinked to one another. By “hydrogel” or “hydrogel network” is meant a network of polymer chains that are water-insoluble, sometimes found as a colloidal gel in which water is the dispersion medium. In other words, hydrogels are a class of polymeric materials that can absorb large amounts of water without dissolving. Hydrogels can contain over 99% water and may comprise natural or synthetic polymers, or a combination thereof. Hydrogels may also possess a degree of flexibility very similar to natural tissue, due to their significant water content. By “hydrogel subunits” or “hydrogel precursors” refers to hydrophilic monomers, prepolymers, or polymers that can be crosslinked, or “polymerized”, to form a 3D hydrogel network. Without being bound by any scientific theory, fixation of the biological sample in the presence of hydrogel subunits may crosslink the components of the biological sample to the hydrogel subunits, thereby securing molecular components in place, preserving the tissue architecture and cell morphology.

In some cases, the biological sample (e.g., cell or tissue) may be permeabilized or otherwise made accessible to an environment external to the biological sample. In some cases, the biological sample may be fixed and permeabilized first, and then a matrix-forming material can then be added into the biological sample.

Any suitable biological sample that comprises nucleic acid may be obtained from a subject. Any suitable biological sample that comprises nucleic acid may be used in the methods and systems described herein. A biological sample may be solid matter (e.g., biological tissue) or may be a fluid (e.g., a biological fluid). In general, a biological fluid can include any fluid associated with living organisms. Non-limiting examples of a biological sample include blood (or components of blood—e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, microbiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues. A biological sample may be a cell-free sample. Such cell-free sample may include DNA and/or RNA.

Any convenient fixation agent, or “fixative,” may be used to fix the biological sample in the absence or in the presence of hydrogel subunits, for example, formaldehyde, paraformaldehyde, glutaraldehyde, acetone, ethanol, methanol, etc. In some cases, the fixative may be diluted in a buffer, e.g., saline, phosphate buffer (PB), phosphate buffered saline (PBS), citric acid buffer, potassium phosphate buffer, etc., usually at a concentration of about 1-10%, e.g. 1%, 2%, 3%, 4%, 5%, 6%, 7%, 8%, or 10%, for example, 4% paraformaldehyde/0.1M phosphate buffer; 2% paraformaldehyde/0.2% picric acid/0.1M phosphate buffer; 4% paraformaldehyde/0.2% periodate/1.2% lysine in 0.1 M phosphate buffer; 4% paraformaldehyde/0.05% glutaraldehyde in phosphate buffer; etc. The type of fixative used and the duration of exposure to the fixative will depend on the sensitivity of the molecules of interest in the specimen to denaturation by the fixative, and may be readily determined using histochemical or immunohistochemical techniques.

The fixative/hydrogel composition may comprise any hydrogel subunits, such as, but not limited to, poly(ethylene glycol) and derivatives thereof (e.g. PEG-diacrylate (PEG-DA), PEG-RGD), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose and the like. Agents such as hydrophilic nanoparticles, e.g., poly-lactic acid (PLA), poly-glycolic acid (PLG), poly(lactic-co-glycolic acid) (PLGA), polystyrene, poly(dimethylsiloxane) (PDMS), etc. may be used to improve the permeability of the hydrogel while maintaining patternability. Materials such as block copolymers of PEG, degradable PEO, poly(lactic acid) (PLA), and other similar materials can be used to add specific properties to the hydrogel. Crosslinkers (e.g. bis-acrylamide, diazirine, etc.) and initiators (e.g. azobisisobutyronitrile (AIBN), riboflavin, L-arginine, etc.) may be included to promote covalent bonding between interacting macromolecules in later polymerization operations.

The biological sample (e.g., a cell or tissue) may be permeabilized after being fixed. Permeabilization may be performed to facilitate access to cellular cytoplasm or intracellular molecules, components or structures of a cell. Permeabilization may allow an agent (such as a phospho-selective antibody, a nucleic acid conjugated antibody, a nucleic acid probe, a primer, etc.) to enter into a cell and reach a concentration within the cell that is greater than which can normally penetrate into the cell in the absence of such permeabilizing treatment. In some embodiments, cells may be stored following permeabilization. In some cases, the cells may be contacted with one or more agents to allow penetration of the one or more agent after permeabilization without any storage and then analyzed. In some embodiments, cells may be permeabilized in the presence of at least about 60%, 70%, 80%, 90% or more methanol (or ethanol) and incubated on ice for a period of time. The period of time for incubation can be at least about 10, 15, 20, 25, 30, 35, 40, 50, 60 or more minutes.

In some embodiments, permeabilization of the cells may be performed by any suitable method. Selection of an appropriate permeabilizing agent and optimization of the incubation conditions and time may be performed. Suitable methods include, but are not limited to, exposure to a detergent (such as CHAPS, cholic acid, deoxycholic acid, digitonin, n-dodecyl-beta-D-maltoside, lauryl sulfate, glycodeoxycholic acid, n-lauroylsarcosine, saponin, and triton X-100) or to an organic alcohol (such as methanol and ethanol). Other permeabilizing methods can comprise the use of certain peptides or toxins that render membranes permeable. Permeabilization may also be performed by addition of an organic alcohol to the cells.

Permeabilization can also be achieved, for example, by way of illustration and not limitation, through the use of surfactants, detergents, phospholipids, phospholipid binding proteins, enzymes, viral membrane fusion proteins and the like; through the use of osmotically active agents; by using chemical crosslinking agents; by physicochemical methods including electroporation and the like, or by other permeabilizing methodologies.

Thus, for instance, cells may be permeabilized using, for example, exposure to one or more detergents (e.g., digitonin, Triton X-100™, NP-40™, octyl glucoside and the like) at concentrations below those used to lyse cells and solubilize membranes (e.g., below the critical micelle concentration). Certain transfection reagents, such as dioleoyl-3-trimethylammonium propane (DOTAP), may also be used. ATP can also be used to permeabilize intact cells. Low concentrations of chemicals used as fixatives (e.g., formaldehyde) may also be used to permeabilize intact cells.

The biological sample within the 3D matrix may be cleared of proteins and/or lipids that are not targets of interest. For example, the biological sample can be cleared of proteins (also called “deproteination”) by enzymatic proteolysis. The clearing may be performed before or after covalent immobilization of any target molecules or derivatives thereof.

In some cases, the clearing is performed after covalent immobilization of target nucleic acid molecules (e.g., RNA or DNA), primers (e.g., RT primers), derivatives of target molecules (e.g., cDNA or amplicons), probes (e.g., padlock probes) to a synthetic 3D matrix. Performing the clearing after immobilization can enable any subsequent nucleic acid hybridization reactions to be performed under conditions where the sample has been substantially deproteinated, as by enzymatic proteolysis (“protein clearing”). This method can have the benefit of removing ribosomes and other RNA- or nucleic-acid-target-binding proteins from the target molecule (while maintaining spatial location), where the protein component may impede or inhibit primer binding, reverse transcription, or padlock ligation and amplification, thereby improving the sensitivity and quantitativity of the assay by reducing bias in nucleic acid hybridization events due to protein occupation of or protein crowding/proximity to the target nucleic acid.

The clearing can comprise removing non-targets from the 3D matrix. The clearing can comprise degrading the non-targets. The clearing can comprise exposing the sample to an enzyme (e.g., a protease) able to degrade a protein. The clearing can comprise exposing the sample to a detergent.

Proteins may be cleared from the sample using enzymes, denaturants, chelating agents, chemical agents, and the like, which may break down the proteins into smaller components and/or amino acids. These smaller components may be easier to remove physically, and/or may be sufficiently small or inert such that they do not significantly affect the background. Similarly, lipids may be cleared from the sample using surfactants or the like. In some cases, one or more of these agents are used, e.g., simultaneously or sequentially. Non-limiting examples of suitable enzymes include proteinases such as proteinase K, proteases or peptidases, or digestive enzymes such as trypsin, pepsin, or chymotrypsin. Non-limiting examples of suitable denaturants include guanidine HCl, acetone, acetic acid, urea, or lithium perchlorate. Non-limiting examples of chemical agents able to denature proteins include solvents such as phenol, chloroform, guanidinium isocyananate, urea, formamide, etc. Non-limiting examples of surfactants include Triton X-100 (polyethylene glycol p-(l, 1,3,3-tetramethylbutyl)-phenyl ether), SDS (sodium dodecyl sulfate), Igepal CA-630, or poloxamers. Non-limiting examples of chelating agents include ethylenediaminetetraacetic acid (EDTA), citrate, or polyaspartic acid. In some embodiments, compounds such as these may be applied to the sample to clear proteins, lipids, and/or other components. For instance, a buffer solution (e.g., containing Tris or tris(hydroxymethyl)aminomethane) may be applied to the sample, then removed.

In some cases, nucleic acids that are not target of interest may also be cleared. These non-target nucleic acids may not be captured and/or immobilized to the 3D matrix, and therefore can be removed with an enzyme to degrade nucleic acid molecules. Non-limiting examples of DNA enzymes that may be used to remove DNA include DNase I, dsDNase, a variety of restriction enzymes, etc. Non-limiting examples of techniques to clear RNA include RNA enzymes such as RNase A, RNase T, or RNase H, or chemical agents, e.g., via alkaline hydrolysis (for example, by increasing the pH to greater than 10). Non-limiting examples of systems to remove sugars or extracellular matrix include enzymes such as chitinase, heparinases, or other glycosylases. Non-limiting examples of systems to remove lipids include enzymes such as lipidases, chemical agents such as alcohols (e.g., methanol or ethanol), or detergents such as Triton X-100 or sodium dodecyl sulfate. In this way, the background of the sample may be removed, which may facilitate analysis of the nucleic acid probes or other targets, e.g., using fluorescence microscopy, or other techniques as described herein.

### Three-Dimensional Matrix

The compositions and methods provided herein can be used in in situ sequence, for example, fluorescent in situ sequencing (FISSEQ). In FISSEQ, a biological sample having target nucleic acid molecules may be embedded within a three-dimensional (3D) matrix. The 3D matrix may comprise a plurality of nucleic acids. The 3D matrix may comprise a plurality of nucleic acids covalently or non-covalently attached thereto. The 3D matrix can be a gel matrix. The 3D matrix can be a hydrogel matrix. The 3D matrix can preserve an absolute or relative 3D position of the plurality of nucleic acid molecules.

In some cases, a matrix-forming material may be used to form the 3D matrix. The matrix-forming material may be polymerizable monomers or polymers, or cross-linkable polymers. The matrix-forming material may be polyacrylamide, acrylamide monomers, cellulose, alginate, polyamide, agarose, dextran, or polyethylene glycol. The matrix-forming materials can form a matrix by polymerization and/or crosslinking of the matrix-forming materials using methods specific for the matrix-forming materials and methods, reagents and conditions. The matrix-forming material may form a polymeric matrix. The matrix-forming material may form a polyelectrolyte gel. The matrix-forming material may form a hydrogel gel matrix.

The matrix-forming material may form a 3D matrix including the plurality of nucleic acids while maintaining the spatial relationship of the nucleic acids. In this aspect, the plurality of nucleic acids can be immobilized within the matrix material. The plurality of nucleic acids may be immobilized within the matrix material by co-polymerization of the nucleic acids with the matrix-forming material. The plurality of nucleic acids may also be immobilized within the matrix material by crosslinking of the nucleic acids to the matrix material or otherwise cross-linking with the matrix-forming material. The plurality of nucleic acids may also be immobilized within the matrix by covalent attachment or through ligand-protein interaction to the matrix.

The matrix can be porous thereby allowing the introduction of reagents into the matrix at the site of a nucleic acid for amplification of the nucleic acid. A porous matrix may be made according to various methods. For example, a polyacrylamide gel matrix can be co-polymerized with acrydite-modified streptavidin monomers and biotinylated DNA molecules, using a suitable acrylamide:bis-acrylamide ratio to control the cross-linking density. Additional control over the molecular sieve size and density can be achieved by adding additional cross-linkers such as functionalized polyethylene glycols.

The 3D matrix may be sufficiently optically transparent or may have optical properties suitable for standard sequencing chemistries and deep three-dimensional imaging for high throughput information readout. Examples of the sequencing chemistries that utilize fluorescence imaging include ABI SoLiD, in which a sequencing primer on a template is ligated to a library of fluorescently labeled octamers with a cleavable terminator. After ligation, the template can then be imaged using four color channels (FITC, Cy3, Texas Red and Cy5). The terminator can then be cleaved off leaving a free-end to engage in the next ligation-extension cycle. After all dinucleotide combinations have been determined, the images can be mapped to the color code space to determine the specific base calls per template. The workflow can be achieved using an automated fluidics and imaging device (e.g., SoLiD 5500 W Genome Analyzer). Another example of sequencing platform uses sequencing by synthesis, in which a pool of single nucleotide with a cleavable terminator can be incorporated using DNA polymerase. After imaging, the terminator can be cleaved and the cycle can be repeated. The fluorescence images can then be analyzed to call bases for each DNA amplicons within the flow cell (e.g., HiSeq).

### Sequencing Scheme

The signal amplification methods can be used with sequencing by hybridization or sequencing by ligation. For sequence determination, the methods described herein can be repeated multiple rounds to determine a sequence along the target nucleic acid molecule.

An example sequencing mechanism of sequence by ligation is provided herein. A portion (e.g., the template hybridizing sequence) of nucleic acid probe can be hybridized to a single-stranded template or target and covalently linked to the sequencing primer by a DNA ligase. The template nonhybridizing sequence can include a signal (e.g., one or more detectable labels) which corresponds to one or more known nucleotides in the nucleic acid probe. One such nucleotide can be the terminal hybridized nucleotide in the nucleic acid probe. A set of nucleic acid probes can include an A, C, G, or T as the terminal hybridized nucleotide with a different signal corresponding to one of A, C, G, or T. Since the signal corresponds to a known nucleotide, detection of the signal confirms hybridization and/or ligation of a particular nucleic acid probe from within the set and the identity of the terminal hybridized nucleotide of the oligonucleotide probe. This approach may be used for any nucleotide within the oligonucleotide probe and is not limited to the terminal hybridized nucleotide.

In one example, the template hybridizing sequence can be 6 nucleotides (e.g., N1, N2, N3, N4, N5 and N6) in length, which is complementary with 6 nucleotides of the template or target. If N1 is to be detected, the template nonhybridizing sequence can correspond to the identity of N1 and the rest of the nucleotides (e.g., N2-N6) can be randomly synthesized such that one species of a pool of nucleic acid probes can have the sequence complementary to the 6 nucleotides of the template or target. Upon binding of a sequencing primer, a first nucleic acid probe which is ligated to the sequencing primer can be used to identify the nucleotide at N1, the second nucleic acid probe which is subsequently ligated to the first nucleic acid probe can be used to detect the nucleotide at N1+6 position, and the cycles can be repeated to identify a nucleotide at a position 6 nucleotides from the previous nucleotide.

In some embodiments, the template hybridizing sequence comprises a combination of canonical and non-canonical nucleobases. In DNA, the canonical DNA bases include A, C, G, and T. An example of a non-canonical base includes inosine, which can pair with any canonical base. The length and composition of the template hybridizing sequence can vary. Thus, in some embodiments, the template hybridizing sequence comprises 3, 4, 5, 6, 7, or 8 canonical nucleobases and 3, 4, 5, 6, 7, or 8 non-canonical nucleobases, such as inosine.

The template hybridizing sequence can also comprise a cleavable linkage between two of the bases. For example, a silver nitrate/MESNA compound can be used to cleave a 3′ bridging phosphorothioate linkage to generate a new 5′ phosphate. The position of the cleavable linkage can be varied within the template hybridizing sequence. Examples of template hybridizing sequences include NNNII*III, NNNNN*NII, NNNNN*III, NNNNN*IIIII, wherein N represents a natural nucleic acid, * represents the cleavable moiety, and I represents a non-canonical base like inosine. In these examples, the template hybridizing sequence comprise between 8 and 10 total bases.

For nucleic acid probes having 6 nucleotides in the template hybridizing sequence, a total of 24 sets of nucleic acid readout domains (4 colors*6 positions) may be provided for sequencing. Moreover, the signal (e.g., detectable labels) for a given set of nucleic acid probes can be one of four fluorophores, such that each set can be detected in four different colors of the electromagnetic spectrum, and can be later associated to one of each nucleotide (e.g., A green, C orange, G blue or T red). Alternatively, a two-color scheme can be used where each base (A, C, G, or T) can correspond to, for example, color X (e.g. red), color Y (e.g. green) a combined signal of colors X and Y (e.g. red+green) or a dark signal. In some embodiments, use of a two-color scheme can allow for the simultaneous sequencing of two nucleotide positions using four channels (e.g. red, green, blue, and yellow channels). Each nucleic acid probe set can be prepared as an equal molar ratio before use (e.g., 1 μM each). The template or target may be interrogated in a serial way (e.g., from N1 to N6), but need not be. The set can be hybridized to the template or target and covalently joined to sequencing primer by a DNA ligase. Then, amplification reaction can be carried out with four sets of nucleic acid amplifiers, each set generating a different amplification product that carries or can be bound to a different signal. Each set of the nucleic acid amplifiers can be designed such that it is specifically related to the identification of one of the nucleotides N.

Upon detection, each color can be associated to a nucleotide (e.g., A, C, G or T) at position N1. For example, if on a given template the green spectrum is detected, an A at N1 is identified and correspondingly the complementary paired base T on the template or target can be identified. After detection, the amplification product or the detectable labels can be removed or rendered undetectable. Afterward the template nonhybridizing sequence can be separated from the template hybridizing sequence. The second round of ligation can use the same sets of nucleic acid amplifiers that is used in the first round, as described above. This can allow identifying N1+6 on the template or target. The cleavage, ligation and hybridization series can be repeated as many times as needed to identify N1+12 after the 3rd series, N1+18 after the 4th series, and so on. Afterward, this extended sequencing primer can be stripped from the template or target.

To identify the complementary nucleotide of N2 on the template or target, sequencing primer can be hybridized to the target nucleic acid molecule. A different set of four nucleic acid probes can be ligated, for example, the set of nucleic acid probes designed to identify N2. Identification is performed as described above. This can allow identification of template nucleotide complementary to N2. The series of cleavage, ligation, hybridization and detection can then be repeated using this set of nucleic acid probes to identify nucleotides complementary to N2+6 on the template. This can be repeated as many times as needed, allowing the identification of N2+12 after the 3rd series, N2+18 after the 4th series, and so on. Stripping and probing can be repeated to serially identify the remaining N3 to N6 positions of the template hybridizing sequence and their corresponding nucleotides on the template or target.

Identification of N1 to N6 series of nucleotides can be achieved by using a same set of nucleic acid probes but with a different sequencing primer during each interrogation. The sequencing primer can have one terminal nucleotide removed from the sequencing primer used in the previous interrogation. For the nucleic acid probes having 6 nucleotides in the template hybridizing sequence, one set of nucleic acid probes comprising 4⁶ species may be provided for sequencing.

In some embodiments, the methods comprise the use of additive color systems, wherein for example, two bases from the template-hybridizing region are detected. Such methods can include detecting a first signal from a first probe comprising a first color from a first cycle. Rather than removing the first probe after the first cycle, a second probe is added during a second cycle. The second signal can then be determined using computational signal processing methods wherein the first signal is detected, then the composite first and second signal is detected, and the second signal is computed from the signals detected in the first cycle and the composite-signal cycle. This additive signal strategy may be scaled up to the number of signals encoded by the plurality of template-hybridizing regions and cognate secondary detection domains for each sequencing template, e.g. rolony. In some embodiments, the additive color-design is employed together with alternative fluorescence encoding designs. For example, N1 and N2 can be detected in a first cycle, wherein N1 uses two colors (such as red/green/red+green/no signal) and N2 uses two additional colors (such as yellow/blue/yellow+blue/no signal). Bases N3 and N4 can then be detected in a second cycle using the same 2-base×2-color design without removing the signal from the first cycle.

### Computer Systems

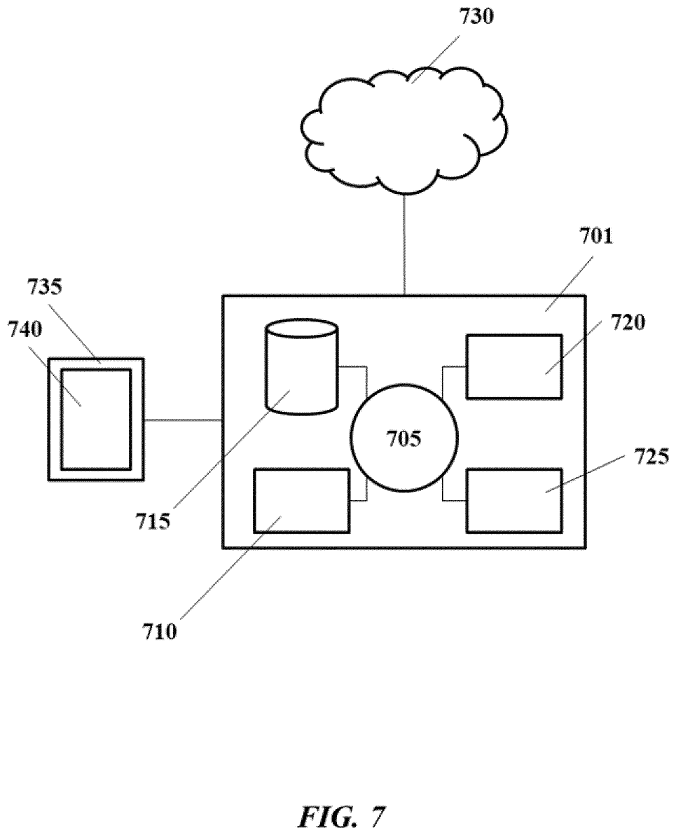

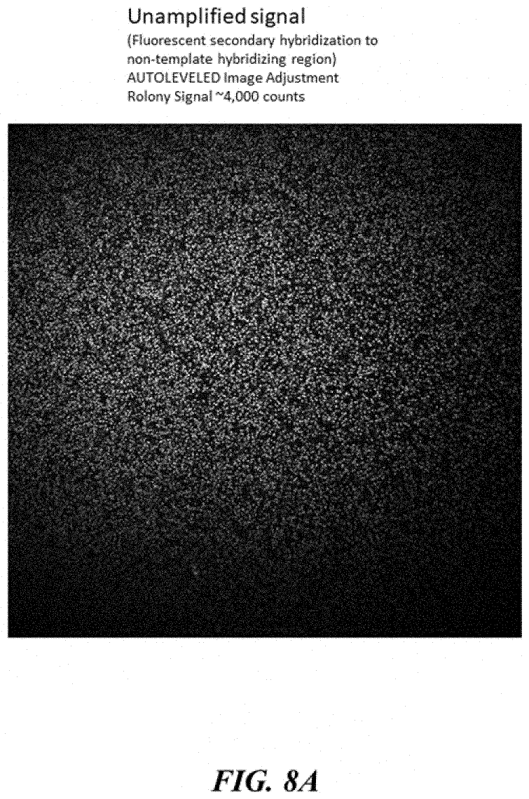

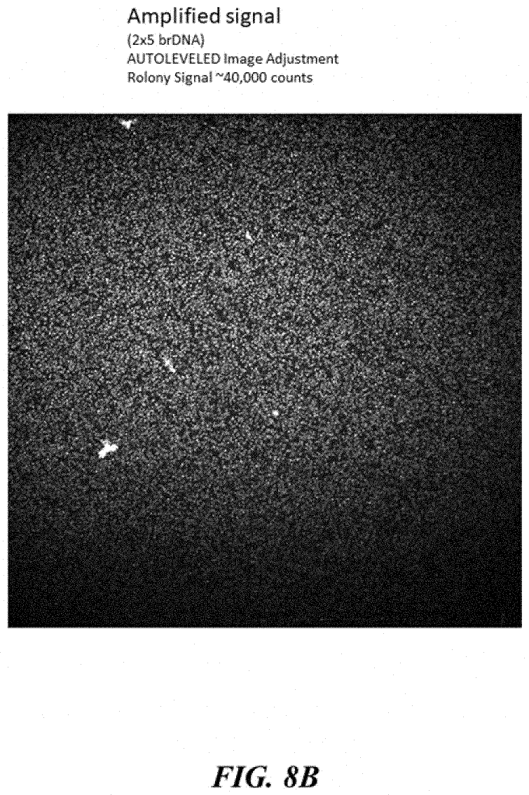

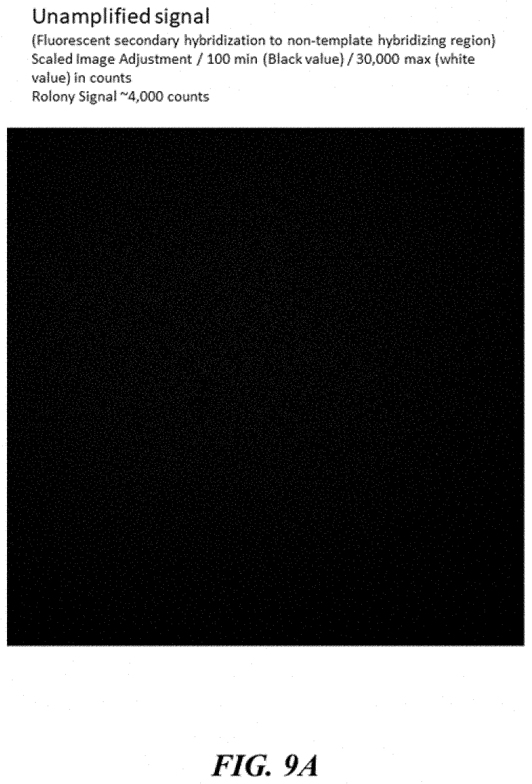

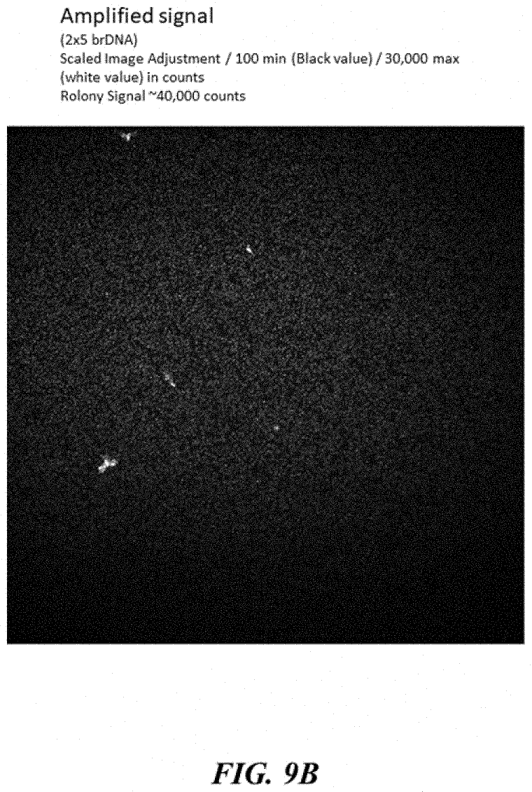

The present disclosure provides computer systems that are programmed to implement methods of the disclosure. FIG. 7 shows a computer system 701 that is programmed or otherwise configured to process a sample using the methods of the present disclosure. The computer system 701 can regulate various aspects of sample processing of the present disclosure, such as, for example, providing a sample in a sample holder, contacting a reagent or buffer to the sample, performing a reaction within the sample and sequencing. The computer system 701 can be an electronic device of a user or a computer system that is remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet. The network 730 in some cases is a telecommunication and/or data network. The network 730 can include one or more computer servers, which can enable distributed computing, such as cloud computing. The network 730, in some cases with the aid of the computer system 701, can implement a peer-to-peer network, which may enable devices coupled to the computer system 701 to behave as a client or a server.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage unit 715 can store files, such as drivers, libraries and saved programs. The storage unit 715 can store user data, e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user (e.g., a user performing sample processing or nucleic acid sequence detection of the present disclosure). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine-readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 can include or be in communication with an electronic display 735 that comprises a user interface (UI) 740 for providing for example, protocols to perform the sample processing methods and/or nucleic acid sequence detection methods described in the present disclosure. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 705. The algorithm can, for example, be executed so as to process a sample and/or detect a nucleic acid sequence utilizing methods and systems disclosed in the present disclosure.

### EXAMPLES

### Example 1—Sequencing by Ligation with Secondary Amplification by Hybridization Chain Reaction

Hybridization chain reaction is used to amplify fluorescent signals that correspond to nucleotides being identified via a sequencing by ligation reaction. Throughout the sequencing by ligation and hybridization chain reaction processes, amplified signals corresponding to identified nucleotides are detected via fluorescent microscopy.

Primary Probe structure: Primary probes of the following structures are used—3′-NNNNN*III-[linker]-[initiator(s)], or 5′-PO4-NNNNN*III-[linker]-[initiator(s)], where N are natural template-hybridizing DNA bases, one or more of which are encoded by the initiator motif, * indicates a bridging phosphorothioate linkage, which is cleaved by silver to enable continuing second strand extension, and “I” represents inosine bases, which act as universal bases but are template-hybridizing (giving an 8-base template hybridizing region). [linker] indicates an optional spacer, e.g., DNA (poly T, poly A, or other) or chemical (e.g., carbon-spacer) to prevent steric inhibition of downstream probing of the initiator motif for secondary amplification.

Sequencing by ligation reaction: To perform sequencing by ligation, 200 μL of 5×SASC is pipetted onto a sample and incubated for 5 minutes at room temperature. Sequencing primer hybridization mix is prepared on ice by mixing 5×SASC buffer and UP2 Minus Seq Primer (UP2_N0 5phos-TCTCGGGTTCGCTGTTGTGGCCTCC; SEQ ID NO: 14) at a ratio of 47:3 (e.g. 188 μL SASC and 12 μL of UP2_N0). Sequencing primer hybridization mix (200 μL) is then pipetted into the sample and the sample is incubated for 30 minutes at room temperature to allow hybridization of the primer to a template nucleic acid (see 202 in FIG. 2). After the 30-minute incubation period, the sequencing primer hybridization mix is removed and the sample is washed via incubation with 200 μL of 1× wash buffer 1E (final concentration of 10 mM tris acetate, 50 mM potassium acetate, 2 mM EDTA, and 0.1% Triton X-100) for 10 minutes. The washing can be repeated for a total of 3 washes.

A sequencing by ligation reaction mix is prepared by mixing water, 10×T4 DNA Ligase Buffer (Enzymatics), a 1 mM solution of primary probes, a 600,000 U/mL T4 DNA Ligase solution (Enzymatics) and a 3,000,000 U/mL T3 DNA Ligase solution (Enzymatics) at a ratio of 167:20:5:4:4. Each primary probe has a structure of—3′-NNNNN*III-[linker]-[initiator(s)], or 5′-PO4-NNNNN*III-[linker]-[initiator(s)] as described above and includes a template hybridizing region and a template non-hybridizing region as shown in 203 of FIG. 2. A mixture of primary probes is used, the mixture including primary probes corresponding to a template strand with A, T, C, or G as the nucleotide in position N1. The final concentration of primary probes in the sequencing by ligation reaction mix is 25 μM. To initiate the sequencing by ligation reaction, samples are incubated in 200 μL of sequencing by ligation reaction mix for 60 minutes at 15° C. while protected from light to allow hybridization and ligation of primary probes that correspond to the identity of the nucleotide at position N1 to the template strand and sequencing primer to the template strand and sequencing primer (see 203 of FIG. 2). Samples then undergo 8 washing rounds with 1× wash buffer 1E to remove primary probes that do not correspond to the nucleotide of the template sequence that is to be identified. Washes 1-3 and 6-8 are 2 minutes each and washes 4 and 5 are 15 minutes each.

Hybridization chain reaction: Following the hybridization of primary probes to the template strand and ligation to sequencing primers (see 202 and 203 of FIG. 2), hybridization chain reaction is used to amplify the signal associated with the hybridized/ligated primary probe, which corresponds to a nucleotide of the template sequence at position N1. To perform hybridization chain reaction, samples with primary probe molecules hybridized to templates and ligated to primers are incubated with 350 μL of amplification buffer (5×SSC, 0.1% Tween 20, and 10% dextran sulfate) for 30 minutes at room temperature. 30 pmol of fluorescently labelled hairpin solutions (containing different hairpins labelled with different fluorescent markers corresponding to each different type of primary probe molecule previously mixed with samples) are prepared by snap cooling (heat at 95° C. for 90 seconds and cool to room temperature on the benchtop for 30 minutes) in 10 μL of 5×SSC buffer. The amplification buffer is then removed. The hairpin solution, which is prepared by adding snap cooled hairpins to 500 μL of amplification buffer at room temperature, is added to samples. Samples are incubated overnight at room temperature during which time the amplification reaction occurs. As can be seen in FIG. 2 and FIG. 6, addition of hairpins that correspond to the non-hybridizing region of primary probe molecules leads to a chain reaction wherein multiple fluorescently labeled DNA strands become indirectly connected to the template strand, with the template non-hybridizing region of the primary probe serving as the initiator of the chain reaction (see 203 of FIG. 2). Hairpin molecules are fluorescently labeled, to correspond to specific primary probes used in the reaction, so that the color of detected fluorescence allows the identification of the hybridized and ligated primary probe. Further, the hybridized and ligated primary probe is specific to the identity of the nucleotide at position N1. Thus, the hybridization chain reaction results in the presence an amplified fluorescent signal that corresponds to the identity of the nucleotide at position N1 of the template strand.

Following the hybridization chain reaction, excess hairpins, which were added but do not correspond to the nucleotide at position N1, are then removed by washing the samples with 500 μL of 5×SSCT (5×SSC and 0.1% Tween 20) at room temperature. Five washes are performed: two 5-minute washes, followed by two 30-minute washes, followed by one 5-minute wash. Amplified fluorescent signals, which correspond to the identity of a nucleotide of the template sequence at a position N1 are detected via fluorescent microcopy.

Following identification of the nucleotide at position N1 of the template strand, the template non-hybridizing (initiator) sequence of the primary probe is separated from the template hybridizing sequence of the primary probe via the addition of a silver nitrate solution to allow the process to be repeated and the identification of additional nucleotides of the template strand (e.g. at N1+5, N1+10, and so on). To identify nucleotides at N2 and other locations, the sequencing primer is stripped from the template, and the entire sequencing by ligation and hybridization chain reaction processes are repeated with primary probes and a sequencing primer designed for the identification of nucleotides at these locations.

### Example 2—Sequencing by Ligation with Secondary Amplification Using Branched Nucleic Acid Amplification

Branched nucleic acid amplification is used to amplify fluorescent signals that correspond to nucleotides being identified via a sequencing by ligation reaction. Throughout the sequencing by ligation and branched nucleic acid amplification processes, amplified signals corresponding to identified nucleotides are detected via fluorescent microscopy.

Primary Probe structure: Primary probes of the following structures are used—3′-NNNNN*III-[linker]-[initiator(s)], or 5′-PO4-NNNNN*III-[linker]-[initiator(s)], where N are natural template-hybridizing DNA bases, one or more of which are encoded by the initiator motif, * indicates a bridging phosphorothioate linkage, which is cleaved by silver to enable continuing second strand extension, and I are inosine bases, which act as universal bases but are template-hybridizing (giving an 8-base template hybridizing region). [linker] indicates the presence of an optional spacer, e.g., DNA (poly T, poly A, or other) or chemical (e.g., carbon-spacer) to prevent steric inhibition of downstream probing of the initiator motif for secondary amplification.

Sequencing by ligation: To perform sequencing by ligation, 200 μL of 5×SASC is pipetted onto a sample and incubated for 5 minutes at room temperature. Sequencing primer hybridization mix is prepared on ice by mixing 5×SASC buffer and UP2 Minus Seq Primer (see Example 1) at a ratio of 47:3 (e.g. 188 μL SASC and 12 μL of UP2_N0). Sequencing primer hybridization mix (200 μL) is then pipetted into the sample and the sample is incubated for 30 minutes at room temperature to allow hybridization of the primer to a template nucleic acid (see 302 in FIG. 3). After the 30-minute incubation period, the sequencing primer hybridization mix is removed and the sample is washed via incubation with 200 μL of 1× wash buffer 1E (final concentration of 10 mM tris acetate, 50 mM potassium acetate, 2 mM EDTA, and 0.1% Triton X-100) for 10 minutes. The washing can be repeated for a total of 3 washes.

A sequencing by ligation reaction mix is prepared by mixing water, 10×T4 DNA Ligase Buffer (Enzymatics), a 1 mM solution of primary probes, a 600,000 U/mL T4 DNA Ligase solution (Enzymatics) and a 3,000,000 U/mL T3 DNA Ligase solution (Enzymatics) at a ratio of 167:20:5:4:4. Each primary probe has a structure of—3′-NNNNN*III-[linker]-[initiator(s)], or 5′-PO4-NNNNN*III-[linker]-[initiator(s)] as described above and includes a template hybridizing region and a template non-hybridizing region as shown in 303 of FIG. 3. A mixture of primary probes is used, the mixture including primary probes corresponding to a template strand with A, T, C, or G as the nucleotide in position N1. The final concentration of nucleic acid probes in the sequencing by ligation reaction mix is 25 μM. To initiate the sequencing by ligation reaction, samples are incubated in 200 μL of sequencing by ligation reaction mix for 60 minutes at 15° C. while protected from light to allow hybridization and ligation of primary probes that correspond to the identity of the nucleotide at position N1 to the template strand and sequencing primer (see 303 of FIG. 3). Samples then undergo 8 washings with 1× wash buffer 1E to remove primary probes that do not correspond to the nucleotide of the template sequence that is to be identified. Washes 1-3 and 6-8 are 2 minutes each and washes 4 and 5 are 15 minutes each.

Branched nucleic acid amplification: Following the hybridization of a primary probe to the template strand and ligation to sequencing primers (see 302 and 303 of FIG. 3), branched nucleic acid amplification is used to amplify a signal associated with the primary probe which corresponds to a nucleotide of the template sequence at position N1.

Preamplifier hybridization mix is prepared by mixing water, 20×SSC, formamide, 45% polyacrylic acid (PAA; MW=8000), and 10 μM of preamplifier in a 108:20:20:40:12 ratio to give a mix with final concentrations of 2×SSC, 10% formamide, 0.05% PAA [MW=8000], and 0.12 nmol preamplifier. The preamplifier hybridization mix contains multiple preamplifier molecules, which each different type of preamplifier molecule corresponding to each of the different primary probe molecules mixed with the sample. To perform branched nucleic acid amplification, samples are incubated in 200 μL of preamplifier hybridization mix in a humidified chamber at 37° C. for 10 minutes to 24 hours to allow hybridization of preamplifier molecules (which correspond to the hybridized primary probe molecule; see 304 in FIG. 3) to the template non-hybridizing region of primary probe molecules. Samples are then washed twice with wash buffer 1E. for 10 minutes per wash at room temperature (wash round 1) to remove unbound preamplifier molecules, which do not correspond to the hybridized and ligated primary probe molecules. Samples are then incubated with 200 μL of amplifier hybridization mix (2×SSC, 10% formamide, 0.05% PAA, 0.6 nmol amplifier) for 10 minutes to 24 hours at 37° C. in a humidified chamber to allow hybridization of amplifier molecules (see 305 in FIG. 3) to the preamplifier molecules. Amplifier hybridization mix contains multiple types of amplifier molecules, each type of amplifier molecule corresponding to a different preamplifier molecule that was previously added to the sample. The amplifier molecules which hybridize to the preamplifier molecules are those that correspond to the preamplifier molecules.

Following hybridization of amplifiers to preamplifiers, samples are washed twice with branched DNA probe wash buffer for 10 minutes per wash at room temperature (wash round 2). Following wash round 2, samples are incubated in 200 μL of base specific reporter probe hybridization mix (2×SSC, 10% formamide, 0.05% PAA, 3 nmol base specific reporter) for 10 minutes to 24 hours at 37° C. in a humidified chamber to allow hybridization of base specific reporter molecules (See 306/307 of FIG. 3). Multiple different types of base specific reporter molecules are added, with each type corresponding to the different amplifier molecules previously added. Each type of base specific reporter probe molecule is labelled with a different fluorescent moiety to correspond to the identity of the hybridized amplifier molecules. Hybridized amplifier molecules correspond to specific hybridized preamplifier molecules, which correspond to specific hybridized and ligated primary probe molecules. Primary probe molecules correspond to the identity of the nucleotide at position N1. Thus, the branched nucleic acid amplification reaction results in the presence an amplified fluorescent signal that corresponds to the identity of the nucleotide at position N1 of the template strand. Samples are then washed twice with branched DNA probe wash buffer for 10 minutes per wash at room temperature and twice more with 2×SSC for 5 minutes per wash to remove unbound base specific reporter molecules that do not correspond to hybridized amplifiers. Amplified fluorescent signals resulting from the hybridized base specific reporter probes are detected via fluorescent imaging.

Following detection of amplified fluorescent signals, the template non-hybridizing (initiator) sequence of the primary probe is then separated from the template hybridizing sequence of the primary probe via the addition of a silver nitrate solution to allow the process to be repeated and for the identification of additional nucleotides of the template strand (e.g. at N1+5, N1+10, and so on). To identify nucleotides at N2 and other locations, the sequencing primer is stripped from the template, and the entire sequencing by ligation and branched nucleic acid amplification processes are repeated with primary probes and a sequencing primer designed for the identification of nucleotides at these locations.

### Example 3—Sequencing by Ligation with Secondary Amplification Using Hybridization Chain Reaction to Detect the Identity and Location of Nucleotides in Tumor Biopsy Samples

A tumor biopsy is taken from a subject and fixed using 4% formaldehyde overnight, followed by 3 washes (including on overnight wash) with 70% EtOH. The sample is washed using PBS and cross-linked using 100 μM BS(PEG)9 (Thermo-Fisher Scientific) in PBS for 1 hour, followed by 1M Tris treatment for fifteen minutes to generate a 3D matrix within the sample. The biopsy sample is then subjected to the sequencing by ligation and hybridization chain reaction amplification reaction processes described in Example 1. Amplified fluorescent signals are detected via scanning confocal microscopy which allows for the detection of the identity and spatial location of nucleotides in the sample. A computer program then generates a spatial map of detected nucleic acid sequences in the biopsy sample.

### Example 4—Sequencing by Ligation with Secondary Amplification Using Branched Nucleic Acid Amplification to Detect the Identity and Location of Nucleotides in Tumor Biopsy Samples

A tumor biopsy is taken from a subject and fixed using 4% formaldehyde overnight, followed by 3 washes (including on overnight wash) with 70% EtOH. The sample is washed using PBS and cross-linked using 100 μM BS(PEG)9 (Thermo-Fisher Scientific) in PBS for 1 hour, followed by 1M Tris treatment for fifteen minutes to generate a 3D matrix within the sample. The biopsy sample is then subjected to the sequencing by ligation and branched nucleic acid amplification reaction processes described in Example 2. Amplified fluorescent signals are detected via scanning confocal microscopy which allows for the detection of the identity and spatial location of nucleotides in the sample. A computer program then generates a spatial map of detected nucleic acid sequences in the biopsy sample.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

SEQUENCES

| SEQ | | |
|---|---|---|
| ID | | |
| NO: | SEQUENCE | ANNOTATION |
| 1 | GTT CCT CAT TCT CTG AAG ANN NNN NNN NNN NNN NNN | Example |
| | NNN NNN NNN NNN NNN NNN NAC TTC AGC TGC CCC GG | template the N |
| | | portion |
| | | represents a |
| | | ssDNA template |
| | | to be identified, |
| | | GTT CCT CAT |
| | | TCT CTG AAG |
| | | A and AC TTC |
| | | AGC TGC CCC |
| | | GG represent |
| | | adapters that can |
| | | be used as a |
| | | sequencing |
| | | primer |
| | | hybridization |
| | | site |
| 2 | GTT CCT CAT TCT CTG AAG A | Adapter |
| | | sequence |
| 3 | AC TTC AGC TGC CCC GG | Adapter |
| | | sequence |
| 4 | ATGAGGAACCCGGGGCAG | Bridge oligo |
| 5 | AATGAGGAACCCGGGGCA*G*C | RCA primer (* |
| | | represents |
| | | phosphorothioate |
| | | bond) |
| 6 | A*A*TGAGGAACCCGGGGCAGC | RCA primer (* |
| | | represents |
| | | phosphorothioate |
| | | bond) |
| 7 | GTTCCTCATTCTCTGAAGA | Ad1 |
| 8 | TCTTCAGAGAATGAG | Ad2 |
| 9 | CCGGGGCAGCTGAAGT | Ad3 |
| 10 | ACTTCAGCTGCC | Ad4 |
| 11 | GAAGTCTTCTTACTCCTTGGGCCCCGTCAGACTTC | Ad5 |
| 12 | GTTCCGAGATTTCCTCCGTTGTTGTTAATCGGAAC | Ad6 |
| 13 | TAACAACAACGGAGGAAA | |
| 14 | TCTCGGGTTCGCTGTTGTGGCCTCC | UP2 Minus Seq |
| | | Primer |

1.-37. (canceled)

38. A method for identifying one or more nucleotides of a nucleic acid molecule, comprising:
(a) providing said nucleic acid molecule having hybridized thereto a sequencing primer and at least one nucleic acid probe, wherein said at least one nucleic acid probe comprises (i) a template hybridizing sequence that is complementary to a sequence of said nucleic acid molecule and (ii) a template nonhybridizing sequence, which template nonhybridizing sequence corresponds to said one or more nucleotides of said nucleic acid molecule, and wherein said template nonhybridizing sequence comprises a nucleic acid initiator;
(b) ligating said sequencing primer to said at least one nucleic acid probe;
(c) contacting said nucleic acid molecule with a plurality of nucleic acid amplifiers such that said nucleic acid initiator of said template nonhybridizing sequence initiates an amplification reaction of at least a subset of said plurality of nucleic acid amplifiers to form an amplification product attached to said nucleic acid initiator;
(d) detecting a signal from said amplification product to identify one or more other nucleotides of said template nonhybridizing sequence, which one or more other nucleotides corresponds to said one or more nucleotides of said nucleic acid molecule; and
(e) using at least said one or more other nucleotides identified in (d) to identify said one or more nucleotides of said nucleic acid molecule.

39. The method of claim 38, wherein said amplification reaction is a hybridization chain reaction (HCR), and wherein a nucleic acid amplifier of said plurality of nucleic acid amplifiers is an HCR monomer.

40. The method of claim 39, wherein said HCR monomer comprises a detectable label.

41. The method of claim 40, wherein said detectable label is attached to said HCR monomer through a linker.

42. The method of claim 41, wherein said linker is a cleavable linker.

43. The method of claim 42, further comprising, subsequent to (e), cleaving said cleavable linker, thereby cleaving said detectable label from said HCR monomer.

44. The method of claim 38, wherein said amplification reaction is a branched nucleic acid amplification, and wherein said nucleic acid initiator is attached to said amplification product through a preamplifier sequence.

45. The method of claim 44, wherein a nucleic acid amplifier of said plurality of nucleic acid amplifiers comprises a first portion that is complementary to said preamplifier sequence and a second portion that is not hybridizable to said preamplifier sequence.

46. The method of claim 45, wherein said second portion of said nucleic acid amplifier further comprises a detectable label.

47. The method of claim 46, wherein said detectable label is attached to said second portion of said nucleic acid amplifier.

48. The method of claim 46, further comprising contacting said second portion of said nucleic acid amplifier with a probe comprising said detectable label.

49. The method of claim 38, wherein said nucleic acid molecule is in a sample, and wherein (c) or (d) is performed while said nucleic acid molecule is in said sample.

50. The method of claim 49, wherein said sample is a cell or a tissue.

51. The method of claim 50, wherein said sample is fixed or permeabilized.

52. The method of claim 49, wherein said sample is immobilized on a surface.

53. The method of claim 38, wherein said nucleic acid molecule is immobilized on a surface.

54. The method of claim 38, wherein said signal is a fluorescent signal or an optical signal.

55. The method of claim 38, further comprising removing said signal from said amplification product or from said nucleic acid molecule.

56. A composition comprising:
a nucleic acid molecule having hybridized thereto a sequencing primer and at least one nucleic acid probe hybridized thereto, wherein said at least one nucleic acid probe comprises (i) a template hybridizing sequence that is complementary to a sequence of said nucleic acid molecule and (ii) a template nonhybridizing sequence, which template nonhybridizing sequence corresponds to one or more nucleotides of said nucleic acid molecule, wherein said template nonhybridizing sequence comprises a nucleic acid initiator, and wherein said sequencing primer is ligated to said at least one nucleic acid probe; and
a plurality of nucleic acid amplifiers, wherein at least a subset of said plurality of nucleic acid amplifiers is configured to form an amplification product attached to said nucleic acid initiator.

57. A kit for identifying one or more nucleotides of a nucleic acid molecule, comprising:
a sequencing primer;
at least one nucleic acid probe, wherein said at least one nucleic acid probe comprises (i) a template hybridizing sequence that is complementary to a sequence of said nucleic acid molecule and (ii) a template nonhybridizing sequence, which template nonhybridizing sequence corresponds to said one or more nucleotides of said nucleic acid molecule, and wherein said template nonhybridizing sequence comprises a nucleic acid initiator;
a plurality of nucleic acid amplifiers; and
instructions that direct a user to:
(a) provide said nucleic acid molecule having hybridized thereto said sequencing primer and said at least one nucleic acid probe;
(b) ligate said sequencing primer to said at least one nucleic acid probe;
(c) contact said nucleic acid molecule with said plurality of nucleic acid amplifiers such that said nucleic acid initiator of said template nonhybridizing sequence initiates an amplification reaction of at least a subset of said plurality of nucleic acid amplifiers to form an amplification product attached to said nucleic acid initiator;
(d) detect a signal from said amplification product to identify one or more other nucleotides of said template nonhybridizing sequence, which one or more other nucleotides corresponds to said one or more nucleotides of said nucleic acid molecule; and
(e) use at least said one or more other nucleotides identified in (d) to identify said one or more nucleotides of said nucleic acid molecule.


SEQUENCE LISTING (15 sequences)

SEQ ID NO: 1 - DNA, 71 bp, Artificial Sequence
  Description of Artificial Sequence Synthetic oligonucleotide
  modified_base (20)..(55): a, c, t, g, unknown or other
gttcctcattctctgaagannnnnnnnnnnnnnnnnnnnnnnnnnnnnnnnnnnnacttcagctgccccgg

SEQ ID NO: 2 - DNA, 19 bp, Artificial Sequence
  Description of Artificial Sequence Synthetic oligonucleotide
gttcctcattctctgaaga

SEQ ID NO: 3 - DNA, 16 bp, Artificial Sequence
  Description of Artificial Sequence Synthetic oligonucleotide
acttcagctgccccgg

SEQ ID NO: 4 - DNA, 18 bp, Artificial Sequence
  Description of Artificial Sequence Synthetic oligonucleotide
atgaggaacccggggcag

SEQ ID NO: 5 - DNA, 20 bp, Artificial Sequence
  Description of Artificial Sequence Synthetic primer
aatgaggaacccggggcagc

SEQ ID NO: 6 - DNA, 20 bp, Artificial Sequence
  Description of Artificial Sequence Synthetic primer
aatgaggaacccggggcagc

SEQ ID NO: 7 - DNA, 19 bp, Artificial Sequence
  Description of Artificial Sequence Synthetic oligonucleotide
gttcctcattctctgaaga

SEQ ID NO: 8 - DNA, 15 bp, Artificial Sequence
  Description of Artificial Sequence Synthetic oligonucleotide
tcttcagagaatgag

SEQ ID NO: 9 - DNA, 16 bp, Artificial Sequence
  Description of Artificial Sequence Synthetic oligonucleotide
ccggggcagctgaagt

SEQ ID NO: 10 - DNA, 12 bp, Artificial Sequence
  Description of Artificial Sequence Synthetic oligonucleotide
acttcagctgcc

SEQ ID NO: 11 - DNA, 35 bp, Artificial Sequence
  Description of Artificial Sequence Synthetic oligonucleotide
gaagtcttcttactccttgggccccgtcagacttc

SEQ ID NO: 12 - DNA, 35 bp, Artificial Sequence
  Description of Artificial Sequence Synthetic oligonucleotide
gttccgagatttcctccgttgttgttaatcggaac

SEQ ID NO: 13 - DNA, 18 bp, Artificial Sequence
  Description of Artificial Sequence Synthetic oligonucleotide
taacaacaacggaggaaa

SEQ ID NO: 14 - DNA, 25 bp, Artificial Sequence
  Description of Artificial Sequence Synthetic primer
tctcgggttcgctgttgtggcctcc

SEQ ID NO: 15 - DNA, 19 bp, Artificial Sequence
  Description of Artificial Sequence Synthetic primer
acggggcccaaggagtaag